(19) United States
(12) Patent Application Publication
(10) Pub. No.: US 2022/0125928 A1
(43) Pub. Date: Apr. 28, 2022

(54) COMPOSITIONS AND METHODS FOR STABILIZING PROTEIN-CONTAINING FORMULATIONS

(71) Applicant: Genentech, Inc., South San Francisco, CA (US)

(72) Inventors: Yilma T. Adem, South San Francisco, CA (US); Lance J. Cadang, South San Francisco, CA (US)

(73) Assignee: Genentech, Inc., South San Francisco, CA (US)

(21) Appl. No.: 17/490,877

(22) Filed: Sep. 30, 2021

Related U.S. Application Data
(63) Continuation of application No. PCT/US2020/025683, filed on Mar. 30, 2020.
(60) Provisional application No. 62/827,402, filed on Apr. 1, 2019.

(51) Int. Cl. A61K 47/26; C07K 16/28; A61K 9/19

(52) U.S. Cl. CPC A61K 47/26; A61K 9/19; C07K 16/2803

(57) ABSTRACT

The present invention relates to use of certain cholate surfactant comprising compositions for enhancing the storage stability of antibodies and other proteins in therapeutically useful formulations.

### CROSS-REFERENCE TO RELATED APPLICATIONS

This application is a continuation of International Application No. PCT/US2020/025683, filed Mar. 30, 2020, which claims the benefit of priority of U.S. Provisional Application No. 62/827,402, filed Apr. 1, 2019, each of which is incorporated by reference herein in its entirety for any purpose.

### FIELD

The present invention relates to use of certain cholate surfactant comprising compositions for enhancing the storage stability of antibodies and other proteins in therapeutically useful formulations.

### BACKGROUND

When a stabilizer for a protein formulation is needed to protect a protein from denaturation upon shaking, agitation, shearing and freeze thaw, or in quiescent state at interface, a nonionic detergent (i.e., a surfactant) is often used (see, e.g., U.S. Pat. No. 5,183,746). This is exemplified by the use of polysorbates in many protein-containing products. For example, polysorbates 20 and 80 (also known as Tween® 20 and Tween® 80) are used in the formulation of biotherapeutic products for both preventing surface adsorption and as stabilizers against protein aggregation (Kerwin, J. Pharm. Sci. 97(8):2924-2936 (2008)). The polysorbates are amphipathic, nonionic surfactants composed of fatty acid esters of polyoxyethylene (POE) sorbitan, being polyoxyethylene sorbitan monolaurate for polysorbate 20 and polyoxyethylene sorbitan monooleate for polysorbate 80.

Unfortunately, polysorbates can undergo degradation via either oxidation or hydrolysis. When a polysorbate molecule degrades, it generates various degradation byproducts including, for example, free fatty acids, POE sorbitan, PEG, PEG esters and alkyl acids. Certain of these byproducts, including the free fatty acids (FFA), can increase turbidity and protein aggregation in protein-containing formulations and may reduce the amount of intact polysorbates that can protect the protein in the formulation from aggregation or oxidation. Therefore, while polysorbates are commonly used as protein stabilizers, the fatty acids and other degradation byproducts released from polysorbate degradation over time can adversely impact the protective effect that polysorbates exhibit in protein-containing formulations.

Proteins undergo varying degrees of degradation during purification and storage, wherein oxidation (including, light-induced oxidation) is one of the major degradation pathways that has a destructive effect on protein stability and potency. Oxidative reactions cause destruction of amino acid residues, peptide bond hydrolysis, and hence protein instability due to alteration of the protein's tertiary structure and protein aggregation (Davies, J. Biol. Chem. 262: 9895-901 (1987)). Oxidation of protein pharmaceuticals have been reviewed by Nguyen (Chapter 4 in Formulation and Delivery of Protein and Peptides (1994)), Hovorka, (J. Pharm Sci. 90:25369 (2001)) and Li (Biotech Bioengineering 48:490-500 (1995)).

Given the above, it is evident that there is a need for the identification of compositions useful for enhancing the stability and preventing the aggregation and/or oxidation of proteins in protein-containing formulations.

### SUMMARY OF THE INVENTION

The present disclosure is based upon the novel finding that certain cholate surfactants are useful for stabilizing and/or reducing aggregation of antibodies or other proteins in therapeutically useful formulations and also for reducing the degradation of polysorbate surfactants in such formulations. Furthermore, the cholate surfactants herein may be useful in stabilizing protein-containing therapeutic formulations at concentrations below their critical micelle concentration (CMC) values of at least about 2.0 mM or at least about 0.2% (weight volume, w/v) as protein stabilizing, or aggregation-reducing, agents. In certain embodiments, a cholate-based surfactant may also protect a therapeutic protein formulation more effectively than an alkylglycoside surfactant at concentrations below the CMC value. Accordingly, in one aspect, the present disclosure relates to formulations of proteins, such as proteins intended for therapeutic use that comprise at least one cholate surfactant at a concentration below its CMC value measured in water at 25° C. In certain embodiments, the protein present in the composition of matter is an antibody, which may optionally be a monoclonal antibody. The present disclosure also relates to containers holding such formulations, articles of matter comprising such containers, and methods of preparing the formulations.

In some embodiments, the formulations may be aqueous, may be stable at a temperature of about 2-8° C. for at least one year, and/or may be stable at a temperature of about 30° C. for at least one month. In some embodiments, the formulation comprises no polysorbate or poloxamer. In other embodiments, the formulation comprises polysorbate and/or poloxamer. In some embodiments, the formulation comprises no alkylglycosides. In other embodiments, the formulation comprises alkylglycosides. In some embodiments, the formulation comprises no other surfactants other than cholates. In other embodiments, the formulation comprises other surfactants.

The present disclosure comprises, inter alia, protein formulations comprising a protein and at least one cholate surfactant having a critical micelle concentration (CMC) value of 2.0 mM or greater or of 0.2% (w/v) or greater in water at 25° C. In some embodiments, the protein is an antibody, such as a monoclonal antibody. In some embodiments, the cholate surfactant is zwitterionic, nonionic, anionic, or is selected from CHAPS (3-[(3-cholamidopropyl) dimethylammonio]-1-propanesulfonate), SGH (sodium glycocholate hydrate), sodium taurocholate hydrate (STH), sodium cholate hydrate (SCH), SdTH, SdCH, ScdCH, and BigCHAP (N,N′-bis-(3-D-gluconamidopropyl) cholamide). In some embodiments, the formulation comprises CHAPS at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%. In some embodiments, the formulation comprises CHAPS at a concentration of 0.025% to 0.05% (w/v). In some embodiments, the formulation comprises BigCHAP at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%. In some embodiments, the formulation comprises BigCHAP at a concentration of 0.025% to 0.05% (w/v). In some embodiments, the formulation comprises SGH, STH, or SCH at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%. In some embodiments, the formulation comprises SGH, STH, or SCH at a concentration of 0.025% to 0.05%. In some embodiments, the at least one cholate surfactant is present at a concentration that is lower than its CMC value in water at 25° C.

In some embodiments, the formulation comprises a zwitterionic or nonionic cholate surfactant and is a low ionic strength formulation. In some such cases, the formulation contains less than 50 mM salt, less than 40 mM salt, less than 30 mM salt, or less than 25 mM salt, such as sodium, arginine, or histidine salt.

In some embodiments, the formulation comprises an anionic cholate surfactant and is a high ionic strength formulation. In some such cases, the formulation comprises at least 175 mM salt, at least 200 mM salt, at least 225 mM salt, or at least 250 mM salt, such as sodium, arginine, or histidine salt.

In some embodiments, the formulation is suitable for therapeutic use. In some embodiments, the formulation has not been subjected to lyophilization, such as a ready-to-use, liquid formulation. Alternatively, the formulation is a reconstituted, lyophilized formulation.

In some embodiments, the formulation does not comprise any polysorbate, poloxamer, pluronic, Brij, or alkylglycoside surfactant. In some embodiments, the formulation does not comprise any non-cholate surfactant. In some embodiments, the formulation consists essentially of at least one cholate surfactant, at least one protein species, at least one buffer species, and at least one non-surfactant stabilizer (e.g., a sugar, sugar alcohol, amino acid, peptide, salt, or other protein). In some embodiments, the formulation further comprises at least one polysorbate or poloxamer, such as polysorbate 20 or polysorbate 80. In some embodiments, the formulation comprises 1.0% or less, 0.05% or less, 0.04% or less, 0.025% or less, 0.02% or less, or 0.01% or less of polysorbate 20 or 80. In other embodiments, the formulation does not comprise any surfactant other than the cholate surfactant and the polysorbate 20 or 80.

The present disclosure also includes a therapeutic protein formulation, comprising at least one therapeutic protein species, and a surfactant consisting essentially of CHAPS at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%, and optionally further comprising one or more of a buffer, a salt, a lyoprotectant, or stabilizer comprising one or more of a sugar, sugar alcohol, amino acid, or other protein species, optionally wherein: (a) the formulation is low ionic strength; (b) the at least one therapeutic protein is an antibody; and/or (c) the formulation is a liquid formulation that is not lyophilized prior to use. In some embodiments, the surfactant consists essentially of 0.01 to 0.05% or 0.025% to 0.05% (w/v) CHAPS. In some embodiments, at least one therapeutic protein species, and a surfactant consisting essentially of BigCHAP at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%, and optionally further comprising one or more of a buffer, a salt, a lyoprotectant, or stabilizer comprising one or more of a sugar, sugar alcohol, amino acid, or other protein species, optionally wherein: (a) the formulation is low ionic strength; (b) the at least one therapeutic protein is an antibody; and/or (c) the formulation is a liquid formulation that is not lyophilized prior to use. In some embodiments, the surfactant consists essentially of 0.01 to 0.05% or 0.025% to 0.05% (w/v) BigCHAP.

The present disclosure also includes a therapeutic protein formulation, comprising at least one therapeutic protein species, and a surfactant consisting essentially of STH, SGH, or SCH at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%, wherein the formulation is a high ionic strength formulation, optionally further comprising one or more of a buffer, a salt, a lyoprotectant, or stabilizer comprising one or more of a sugar, sugar alcohol, amino acid, or other protein species, and optionally wherein: (a) the at least one therapeutic protein is an antibody; and/or (b) the formulation is a liquid formulation that is not lyophilized prior to use. In some embodiments, the surfactant consists essentially of 0.01 to 0.05% or 0.025% to 0.05% (w/v) STH, SGH, or SCH.

In some embodiments, the formulation has one or more of the following properties: (a) the formulation shows no visible aggregates after 24 hours of agitation at 100 revolutions per minute (rpm) at room temperature; (b) the formulation shows no more than 2% high molecular weight protein aggregates after 24 hours of agitation at 100 rpm at room temperature; (c) the formulation shows no more than 1% high molecular weight protein aggregates after 24 hours of agitation at 100 at room temperature; (d) high molecular weight protein aggregates in the formulation do not increase by more than 0.2% after 24 hours of agitation at 100 rpm at room temperature compared to a non-agitated control; (e) if the formulation comprises polysorbate 20 or polysorbate 80, the polysorbate 20 or polysorbate 80 in the formulation remains intact to a larger degree after 2 weeks storage at 40° C. or after treatment with Candida antarctica lipase B (CALB, Sigma Aldrich CAS #9001-62-1) lipase than a formulation with the same ingredients and concentrations, but without cholate.

The present disclosure also includes containers comprising the formulations disclosed herein, and articles of manufacture comprising the containers comprising the formulations.

The present disclosure further includes methods of making the protein formulations herein, comprising mixing the protein with the at least one cholate surfactant to form a cholate-containing aqueous solution. The present disclosure also includes methods of inhibiting aggregation of a protein present in an aqueous solution, said method comprising adding to the aqueous solution at least one cholate surfactant having a critical micelle concentration (CMC) value of about 2.0 mM or greater or 0.2% (w/v) in water at 25° C., at a concentration below its CMC value in water at 25° C., to form a cholate-containing aqueous solution. In some such embodiments, the protein is an antibody, such as a monoclonal antibody. In some embodiments, the methods further comprise lyophilizing the cholate-containing aqueous solution. In other embodiments, the methods do not comprise lyophilizing the cholate-containing aqueous solution.

### BRIEF DESCRIPTION OF THE DRAWINGS

FIG. 1 shows results from mixing 0.05% (w/v) of a cholate surfactant or a control surfactant (cholates CHAPS, SGH, or STH, polysorbate 20 (PS20), or poloxamer 188 (PX188)) with an exemplary monoclonal anti-PDL1 antibody at 1 mg/mL in 20 mM histidine acetate and 240 mM sucrose at pH 5.5. Solutions were 5 mL volume in a 15 mL glass vial. Control solutions with the above ingredients but without surfactants were also prepared. The figure shows whether visible aggregates form after agitation for 24 hours at ambient temperature in an arm shaker (Glas-Col bench top arm shaker) at 100 revolutions per minute (rpm).

FIG. 2 shows from mixing an exemplary monoclonal anti-Tryptase antibody at 1 mg/mL in a solution of 200 mM arginine succinate at pH 5.8. Solutions were 5 mL volume in a 15 mL glass vial. Control solutions with the above ingredients but without surfactants were also prepared. The figure shows whether visible aggregates form after agitation for 24 hours at ambient temperature in an arm shaker at 100 rpm.

FIG. 3 shows that cholates can protect free fatty acids in protein solutions from precipitating. Solutions containing 5 mg/mL anti-Tryptase antibody and 200 mM arginine succinate and 0.02% PS20 at pH 5.8 were mixed with various concentrations of a cholate surfactant and then spiked with 0.04 units/mL CALB at 5° C. If cholates protect PS20 from degradation to FFAs, then visible FFA precipitate particles should not form in the protein solutions or such particles, once formed, should re-solubilize upon addition of cholate, while, if cholates provide no protection or solubilization, visible FFA precipitate particles should form to the same degree as protein solutions in which no cholate was added. Results show that addition of 0.5% SCH, SGH, or CHAPS protects against visible particulate formation in the solutions, while such particulates still form at 0.02% to 0.1% of each added surfactant.

FIG. 4 shows a summary of results from incubation of cholate surfactants at different concentrations on PS20 degradation induced by added CALB lipase. The dashed line in the graph provides the PS20 concentration observed upon complete degradation, as shown by the “lipase only” control solution.

### DETAILED DESCRIPTION OF CERTAIN EMBODIMENTS

The present invention may be understood more readily by reference to the following detailed description of specific embodiments and the Examples included below.

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Measured values are understood to be approximate, taking into account significant digits and the error associated with the measurement.

As used herein, percentages (“%”) are weight to volume (“w/v”) percentages unless specified otherwise.

The present disclosure relates to protein and cholate comprising formulations. Such “formulations” may also be interchangeably called “compositions” or “preparations” herein.

In some embodiments herein, a formulation may be of “low ionic strength” or “high ionic strength”. “Ionic strength” represents the strength of the electric field in a solution, and is equal to the sum of the molalities of each type of ion present multiplied by the square of their charges. As used herein, a “low ionic strength” formulation has a salt concentration (e.g. sodium, arginine, histidine, or similar salt) of 50 mM or lower, such as 20 mM to 50 mM. As used herein, a high ionic strength formulation has a salt concentration (e.g. sodium, arginine, histidine or similar) of 150 mM or higher, such as 150 mM to 300 mM.

An “isotonic” formulation is one which has essentially the same osmotic pressure as human blood. Isotonic formulations will generally have an osmotic pressure from about 250 to 350 mOsm. The term “hypotonic” describes a formulation with an osmotic pressure below that of human blood. Correspondingly, the term “hypertonic” is used to describe a formulation with an osmotic pressure above that of human blood. Isotonicity can be measured using a vapor pressure osmometer or freezing point depression osmometer, for example. The formulations of the present disclosure may be hypertonic as a result of the addition of salt and/or buffer.

A “lyophilized” formulation is one that has been freeze-dried or subjected to a lyophilization process. Formulations herein may be lyophilized for storage or alternatively, may be intended for storage as liquid solutions. A “reconstituted” formulation is one that has been prepared by dissolving a lyophilized protein or antibody formulation in a diluent such that the protein is dispersed in the reconstituted formulation. The reconstituted formulation may be suitable for use, such as for administration to a patient to be treated with the protein of interest.

“Surfactants” are molecules with well-defined polar and non-polar regions that allow them to aggregate in solution to form micelles. Depending on the nature of the polar area, surfactants can be non-ionic, anionic, cationic, and zwitterionic.

As used herein, “cholates” or “cholate surfactants” refer to molecules based on the cholic acid backbone, and may be derivatized from cholyl-CoA, becoming functionalized in the conjugation site, and by removal of hydroxyl groups either or both C7 and C12 of the cholate backbone. Cholates herein are a type of surfactant.

“Polypeptide” or “protein” means a sequence of amino acids for which the chain length is sufficient to produce a tertiary structure. Thus, proteins herein are distinguished from “peptides,” which are short amino acid-based molecules that generally do not have any tertiary structure. Typically, a protein for use herein will have a molecular weight of at least about 5-20 kD, alternatively at least about 15-20 kD, preferably at least about 20 kD. Polypeptides or proteins herein include, for example, antibodies.

The term “antibody” as used herein includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antigen-binding fragments (e.g., Fab, F(ab′)2, and Fv). Antibodies herein comprise a set of complementary depending regions (CDRs) located in heavy (H) and light (L) chain variable domains that collectively recognize a particular antigen. Antibodies herein comprise at least the portions of the heavy and light chain variable domain amino acid sequences sufficient to include the set of CDRs for antigen recognition. In some embodiments, antibodies comprise full length heavy and light chain variable domains. In some embodiments, antibodies further comprise heavy and/or light chain constant regions, which may or may not be full length.

The term “immunoglobulin” (Ig) is used interchangeably with “antibody” herein.

The term “pharmaceutical formulation” or “therapeutic formulation” or “therapeutic preparation” refers to a preparation or composition comprising at least one active ingredient (e.g. a protein) and at least one additional component or excipient substance, and which is in such form as to permit the biological activity of the active ingredient to be effective in a mammalian subject, and which is “suitable for therapeutic use” or “suitable for pharmaceutical use,” meaning that the formulation as a whole is not unacceptably toxic to a mammalian subject and does not contain components which are unacceptably toxic to a mammalian subject to which the formulation would be administered or which are at concentrations that would render them unacceptably toxic to a subject.

A “stable” formulation is one in which the protein therein essentially retains its physical and/or chemical stability upon storage. Stability can be measured at a selected temperature for a selected time period. Preferably, the formulation is stable at room temperature (˜30° C.) or at 40° C. for at least 1 month and/or stable at about 2-8° C. for at least 1 year and preferably for at least 2 years. For example, the extent of aggregation during storage can be used as an indicator of protein stability. Thus, a “stable” formulation may be one wherein less than 10% (w/v) and preferably less than 5%, less than 3%, or less than 2% of the protein is present as an aggregate in the formulation. Various analytical techniques for measuring protein stability are available in the art and are reviewed, for example, in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993).

Increasing the “stability” of a protein-containing formulation may involve reducing (as compared to an untreated protein-containing formulation) or preventing the formation of protein aggregates in that formulation or of degradation products of other components of the formulation so that those other components may continue act so as to maintain the stability of the protein.

The term, “stabilizing agent” or “stabilizer” as used herein is a chemical or compound that is added to a formulation to maintain it in a stable or unchanging state. In some cases, a stabilizer may be added to help prevent aggregation, oxidation, color changes, or the like.

The term “aggregate” or “aggregation” as used herein means to come together or collect in a mass or whole, e.g., as in the aggregation of protein molecules. Aggregates can be self-aggregating or aggregate due to other factors, e.g., presence of aggregating agents, precipitating agents, agitation, or other means and methods whereby proteins cause to come together. A protein that is “susceptible to aggregation” is one that has been observed to aggregate with other protein molecules, especially upon agitation. Aggregation may be observed visually, such as when a previously clear protein formulation in solution becomes cloudy or contains precipitates, or by methods such as size exclusion chromatography (SEC), which separates proteins in a formulation by size.

Aggregates may include dimers, trimers, and multimers of the protein species. As used herein, “high molecular weight species” (HMWS) refers to aggregates of proteins that may, for example, be observed by size exclusion chromatography, and that represent at least dimers of the desired protein molecules, i.e., having at least twice the molecular weight of the desired protein species in a formulation. In the case of a protein species such as an antibody that, in its normal or desired form is already a multimer, e.g. a dimer or tetramer, a HMWS would represent at least a dimer of the normal, desired multimeric form of the protein.

By “inhibiting” or “preventing” agitation-induced aggregation is intended to mean preventing, reducing, or decreasing the amount of agitation-induced aggregation, measured by comparing the amount of aggregate present in a protein-containing solution that comprises at least one inhibitor of agitation-induced aggregation with the amount of aggregate present in a protein-containing solution that does not comprise at least one inhibitor of agitation-induced aggregation.

The “critical micelle concentration” (CMC) is the threshold concentration at which a surfactant aggregates in solution to form clusters called micelles. As used herein, CMC values for any particular surfactant are measured at 25° C. in water, and may be expressed in units of mM or percent (w/v). Because the formation of micelles from constituent monomers involves an equilibrium, the existence of a narrow concentration ranges for micelles, below which the solution contains negligible amounts of micelles and above which practically all additional surfactant is found in the form of additional micelles, has been established. A compilation of CMCs for hundreds of compounds in aqueous solution has been prepared by Mukerjee, P. and Mysels, K. J. (1971) Critical Micelle Concentrations of Aqueous Surfactant Systems, NSRDS-NBS 36. Superintendent of Documents, U.S. Government Printing Office, Washington, D.C. See also, http://www.anatrace.com/docs/detergent_data.pdf.

“Isolated” when used to describe the various polypeptides and antibodies disclosed herein, means a polypeptide or antibody that has been identified, separated and/or recovered from a component of its production environment. Preferably, the isolated polypeptide is free of association with all other components from its production environment. Contaminant components of its production environment, such as that resulting from recombinant transfected cells, are materials that would typically interfere with diagnostic or therapeutic uses for the polypeptide, and may include enzymes, hormones, and other proteinaceous or non-proteinaceous solutes. In some embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Ordinarily, however, an isolated polypeptide or antibody will be prepared by at least one purification step.

In some embodiments herein, pharmaceutical formulations “do not comprise” one or more types of excipients or ingredients such as one or more non-cholate surfactants. The expression “does not comprise” in this context means that the excluded ingredients are not present beyond trace levels, for example, due to contamination or impurities found in other purposefully added ingredients.

The term “consisting essentially of” when referring to a mixture of ingredients of a formulation herein indicates that, while ingredients other than those expressly listed may be present, such ingredients are found only in trace amounts or in amounts otherwise low enough that the fundamental characteristics of the formulation including protein concentration, level of protein aggregation, level of protein oxidation, viscosity, thermal stability, osmolality, and pH are unchanged.

Protein Aggregation

Aggregation of proteins is caused mainly by hydrophobic interactions that eventually lead to denaturation. When the hydrophobic region of a partially or fully unfolded protein is exposed to water, this creates a thermodynamically unfavorable situation due to the fact that the normally buried hydrophobic interior is now exposed to a hydrophilic aqueous environment. Consequently, the decrease in entropy from structuring water molecules around the hydrophobic region forces the denatured protein to aggregate, mainly through the exposed hydrophobic regions. Thus, solubility of the protein may also be compromised. In some cases, self-association of protein subunits, either native or misfolded, may occur under certain conditions and this may lead to precipitation and loss in activity.

Factors that affect protein aggregation in solution generally include protein concentration, pH, temperature, other excipients, and mechanical stress. Some factors (e.g., temperature) can be more easily controlled during purification, compounding, manufacturing, storage and use than others (e.g., mechanical stress). Formulation studies will dictate appropriate choice(s) of pH and excipients that will not induce aggregation and/or, in fact, will aid in the prevention of aggregation. Protein concentration is dictated by the required therapeutic dose and, depending on what this concentration is, will determine whether the potential for higher associated states (dimers, tetramers, etc.) exists, which can then lead to aggregation in solution. Careful studies must be done during formulation development to determine what factors influence protein aggregation and then how these factors can be eliminated or controlled.

The desire to identify stable solution preparations of an antibody or other protein for use in parenteral or other administration can lead to the development of test methodology for assessing the impact of various additives on physical stability. Based on the known factors influencing protein aggregation and the requirements of such applications, physical stability may be evaluated using mechanical procedures involving agitation or rotation of protein solutions. The methodology for physical stress testing to identify the capability of various additives to prevent aggregation might involve exposure to shaking or stirring in the horizontal plane or rotation “x” cm from the axis of a wheel rotating at “n” rpm in the vertical plane. Turbidity resulting from aggregation is usually determined as a function of time by visual inspection or light scattering analysis. Alternatively, reductions in the soluble protein content due to precipitation can be quantitated by HPLC assay as a function of time.

Proteins on the surface of water will aggregate, particularly when agitated, because of unfolding and subsequent aggregation of the protein monolayer. Surfactants can denature proteins, but can also stabilize them against surface denaturation. Generally, ionic surfactants can denature proteins. However, nonionic surfactants usually do not denature proteins even at relatively high concentrations of 1% (w/v). The present disclosure is based upon the novel finding that certain cholate surfactants are useful for stabilizing or reducing aggregation of antibodies or other proteins in therapeutically useful formulations.

Cholate Surfactants and Formulations

The present disclosure based upon the novel finding that certain cholate surfactants are useful for stabilizing or reducing aggregation of antibodies or other proteins in therapeutically useful formulations. Exemplary cholates include, but are not limited to, zwitterionic cholates such as CHAPS (3-[(3-cholamidopropyl) dimethylammonio]-1-propanesulfonate) (CAS 75621-03-3), which has a critical micelle concentration (CMC) of about 8-10 mM or 0.5-0.6% in water at 25° C., anionic cholates such as SGH (sodium glycocholate hydrate) (CAS 338950-81-5) (CMC about 13 mM or about 0.6% (w/v) in water at 25° C.), sodium taurocholate hydrate (STH) (CAS 345909-26-4) (CMC about 3-11 mM or about 0.2% to 0.6% in water at 25° C.) and sodium cholate hydrate (SCH) (CMC about 9-15 mM or about 0.4% to 0.7% in water at 25° C.), as well as non-ionic cholates such as “BigCHAP” (N,N′-bis-(3-D-gluconamidopropyl) cholamide) (CAS 86303-22-2) (CMC about 2.9-3.4 mM or about 0.26% in water at 25° C.). In some embodiments, a cholate may have a CMC of at least 1 mM, or at least 2 mM, or at least 0.1% (w/v), or at least 0.2% (w/v), in water at 25° C.

A particular cholate may be employed singly as an antibody or other protein stabilizing agent, or may be employed in combination with other cholates. In particular embodiments of the present invention, the cholate (if employed as a single agent) or cholates (if employed in combination) may be present in the aqueous antibody- or other protein-containing formulation at a concentration from 0.01% to 0.5%, which may be below the CMC values of the cholates employed. In some embodiments, the cholate or cholates may be present at a concentration of 0.5% or less, 0.4% or less, 0.3% or less, 0.2% or less, 0.1% or less, 0.05% or less, 0.04% or less, 0.025% or less, or 0.02% or less. In some embodiments, the cholate or cholates may be present at a concentration from 0.01% to 0.1%, 0.01% to 0.05%, 0.025% to 0.05%, or 0.025% to 0.1%. In some embodiments, the cholate or cholates may be present at a concentration from 0.01% to 0.05%. In some embodiments, the cholate or cholates may be present at a concentration from 0.025% to 0.05%.

In some embodiments, a particular cholate may be employed as an antibody or other protein stabilizing agent at a concentration that is lower than its respective CMC value in water at 25° C. In some embodiments, a cholate may have a CMC of at least 1 mM, or at least 2 mM, or at least 0.1% (w/v), or at least 0.2% (w/v), in water at 25° C. In some embodiments, a mixture of cholates may be employed such that the mixture is at an overall concentration lower than the CMC value of the mixture in water at 25° C. In some such embodiments, the cholate or cholates may be the only type of surfactant present in the composition; thus no other surfactants are present.

Most currently used therapeutically acceptable nonionic surfactants come from either the polysorbate or polyether groups. Polysorbate 20 and 80 are contemporary surfactant stabilizers in marketed therapeutic protein formulations. However, other surfactants used in therapeutic protein formulations include Pluronic® F-68 and members of the “Brij” class and poloxamers and alkylglycosides. In some embodiments herein, none of these other surfactants are present in the formulations, while in other embodiments, one or more of these other classes of surfactants are included.

In some embodiments, the composition does not comprise polysorbates, pluronics, Brij, poloxamer, or alkylglycoside surfactants. In other embodiments, the composition comprises at least one other surfactant. In other embodiments, the composition also comprises one or more polysorbates such as PS20 or PS80 or may comprise an alkylglycoside or combination of alkylglycosides. In some such cases where a formulation comprises a polysorbate surfactant and/or alkylglycoside surfactant, the formulation does not comprise other surfactants beyond the cholate and polysorbate and/or alkylglycoside surfactants.

In some embodiments, the cholate surfactant is CHAPS. In some embodiments, the formulation comprises CHAPS at a concentration (w/v) of 0.5% or less, 0.4% or less, 0.3% or less, 0.2% or less, 0.1% or less, 0.05% or less, 0.04% or less, 0.025% or less, or 0.02% or less. In some embodiments, the CHAPS is present at a concentration from 0.01% to 0.1%, 0.01% to 0.05%, 0.025% to 0.05%, or 0.025% to 0.1%. In some embodiments, the CHAPS is present at a concentration from 0.01% to 0.05%. In some embodiments, the CHAPS is present at a concentration from 0.025% to 0.05%. In some embodiments, the formulation surfactant consists essentially of CHAPS at a concentration of 0.5% or less, 0.4% or less, 0.3% or less, 0.2% or less, 0.1% or less, 0.05% or less, 0.04% or less, 0.025% or less, or 0.02% or less. In some embodiments, the CHAPS is present at a concentration from 0.01% to 0.1%, 0.01% to 0.05%, 0.025% to 0.05%, or 0.025% to 0.1%. In some embodiments, the CHAPS is present at a concentration from 0.01% to 0.05%. In some embodiments, the CHAPS is present at a concentration from 0.025% to 0.05%.

In some embodiments, the formulation comprises at least one therapeutic protein species, and a surfactant consisting essentially of CHAPS at a concentration (w/v) of 0.5% or less, 0.4% or less, 0.3% or less, 0.1% or less, 0.05% or less, 0.04% or less, 0.025% or less, or 0.02% or less, 0.01% to 0.5%, or 0.01% to 0.1%, 0.01% to 0.05%, or 0.025% to 0.05%, and optionally one or more of a buffer, a salt, a lyoprotectant, or stabilizer comprising one or more of a sugar, sugar alcohol, amino acid, or other protein species, optionally wherein: the formulation is low ionic strength; the at least one therapeutic protein is an antibody; and/or the formulation is a liquid formulation that is not lyophilized prior to use. In other embodiments, the formulation further comprises a polysorbate such as PS20 or PS80.

In some embodiments, the cholate surfactant is BigCHAP. In some embodiments, the formulation comprises BigCHAP at a concentration of 0.5% or less, 0.4% or less, 0.3% or less, 0.2% or less, 0.1% or less, 0.05% or less, 0.04% or less, 0.025% or less, or 0.02% or less. In some embodiments, the BigCHAP is present at a concentration from 0.01% to 0.1%, 0.01% to 0.05%, 0.025% to 0.05%, or 0.025% to 0.1%. In some embodiments, the BigCHAP is present at a concentration from 0.01% to 0.05%. In some embodiments, the BigCHAP is present at a concentration from 0.025% to 0.05%. In some embodiments, the formulation surfactant consists essentially of BigCHAP at a concentration of 0.5% or less, 0.4% or less, 0.3% or less, 0.2% or less, 0.1% or less, 0.05% or less, 0.04% or less, 0.025% or less, or 0.02% or less. In some embodiments, the BigCHAP is present at a concentration from 0.01% to 0.1%, 0.01% to 0.05%, 0.025% to 0.05%, or 0.025% to 0.1%. In some embodiments, the BigCHAP is present at a concentration from 0.01% to 0.05%. In some embodiments, the BigCHAP is present at a concentration from 0.025% to 0.05%.

In some embodiments, the formulation comprises at least one therapeutic protein species, and a surfactant consisting essentially of BigCHAP at a concentration of 0.5% or less, 0.4% or less, 0.3% or less, 0.1% or less, 0.05% or less, 0.04% or less, 0.025% or less, or 0.02% or less, 0.01% to 0.5%, or 0.01% to 0.1%, 0.01% to 0.05%, or 0.025% to 0.05%, and optionally one or more of a buffer, a salt, a lyoprotectant, or stabilizer comprising one or more of a sugar, sugar alcohol, amino acid, or other protein species, optionally wherein: the formulation is low ionic strength; the at least one therapeutic protein is an antibody; and/or the formulation is a liquid formulation that is not lyophilized prior to use. In other embodiments, the formulation further comprises a polysorbate such as PS20 or PS80.

In some embodiments, the cholate surfactant is SGH, STH, or SCH. In some embodiments, the formulation comprises SGH, STH, or SCH at a concentration of 0.5% or less, 0.4% or less, 0.3% or less, 0.2% or less, 0.1% or less, 0.05% or less, 0.04% or less, 0.025% or less, or 0.02% or less. In some embodiments, the SGH, STH, or SCH is present at a concentration from 0.01% to 0.1%, 0.01% to 0.05%, 0.025% to 0.05%, or 0.025% to 0.1%. In some embodiments, the SGH, STH, or SCH is present at a concentration from 0.01% to 0.05%. In some embodiments, the SGH, STH, or SCH is present at a concentration from 0.025% to 0.05%. In some embodiments, the formulation surfactant consists essentially of SGH, STH, or SCH at a concentration of 0.5% or less, 0.4% or less, 0.3% or less, 0.2% or less, 0.1% or less, 0.05% or less, 0.04% or less, 0.025% or less, or 0.02% or less. In some embodiments, the SGH, STH, or SCH is present at a concentration from 0.01% to 0.1%, 0.01% to 0.05%, 0.025% to 0.05%, or 0.025% to 0.1%. In some embodiments, the SGH, STH, or SCH is present at a concentration from 0.01% to 0.05%. In some embodiments, the SGH, STH, or SCH is present at a concentration from 0.025% to 0.05%. In some of the above embodiments comprising SGH, STH, or SCH surfactants, the solution has a high ionic strength.

In some embodiments, the formulation comprises at least one therapeutic protein species, and a surfactant consisting essentially of cholate at a concentration (w/v) of 0.5% or less, 0.4% or less, 0.3% or less, 0.1% or less, 0.05% or less, 0.04% or less, 0.025% or less, or 0.02% or less, 0.01% to 0.5%, or 0.01% to 0.1%, 0.01% to 0.05%, or 0.025% to 0.05%, and optionally one or more of a buffer, a salt, a lyoprotectant, or stabilizer comprising one or more of a sugar, sugar alcohol, amino acid, or other protein species, optionally wherein: the formulation is high ionic strength; the at least one therapeutic protein is an antibody; and/or the formulation is a liquid formulation that is not lyophilized prior to use. In other embodiments, the formulation further comprises a polysorbate such as PS20 or PS80.

In some embodiments, the overall formulation has a low ionic strength. A low ionic strength formulation herein may have, for example, a salt concentration (e.g. sodium, acetate, phosphate, arginine, histidine, citrate) of 50 mM or lower, such as 10-50 mM, 20-50 mM, 20-40 mM, 20-30 mM, 15-30 mM, 15-25 mM, 40 mM or lower, 30 mM or lower, 25 mM, or lower, or 20 mM or lower. In some embodiments, for example when using an anionic cholate species, the overall formulation has a high ionic strength. A high ionic strength formulation herein may have 150 mM or higher salt concentration, such as 175 mM or higher, 200 mM or higher, 250 mM or higher, 150-300 mM, 200-300 mM, 200-250 mM, 175-250 mM, or 150-250 mM.

Exemplary Proteins

The present formulations are compatible with a wide variety of proteins or polypeptides.

Examples of polypeptides encompassed within the definition herein include mammalian proteins, such as, e.g., various antibodies, renin; a growth hormone, including human growth hormone and bovine growth hormone; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrand factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin such as human serum albumin; Muellerian-inhibiting substance; relaxin A-chain; relaxin B-chain; prorelaxin; mouse gonadotropin-associated peptide; a microbial protein, such as beta-lactamase; DNase; IgE; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-alpha and TGF-beta, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins (IGFBPs); CD proteins such as CD3, CD4, CD8, CD19 and CD20; erythropoietin; osteoinductive factors; immunotoxins; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor; viral antigen such as, for example, a portion of the AIDS envelope; transport proteins; homing receptors; addressins; regulatory proteins; integrins such as CD11a, CD11b, CD11c, CD18, an ICAM, VLA-4 and VCAM; a tumor associated antigen such as CA125 (ovarian cancer antigen) or HER2, HER3 or HER4 receptor; immunoadhesins; and fragments and/or variants of any of the above-listed proteins as well as antibodies, including antibody fragments, binding to any of the above-listed proteins.

The protein which is formulated is preferably essentially pure and desirably essentially homogeneous (i.e., free from contaminating proteins). “Essentially pure” protein means a composition comprising at least about 90% by weight of the protein, based on total weight of the composition, preferably at least about 95% by weight. “Essentially homogeneous” protein means a composition comprising at least about 99% by weight of protein, based on total weight of the composition.

A protein retains “biological activity” in a pharmaceutical formulation, if the biological activity of the protein at a given time is within about 10% (within the errors of the assay) of the biological activity exhibited at the time the formulation was prepared. In the case of an antibody or a protein that is intended to function by binding to a target molecule or antigen, biological activity may be determined by the ability of the protein in vitro or in vivo to bind to antigen and result in a measurable biological response.

Proteins herein broadly encompass naturally occurring proteins as well as fusion proteins formed, for example, by covalently linking two distinct proteins together, and protein conjugates, which include proteins covalently linked to other proteins or to non-protein molecules such as nucleic acids, small molecule drugs, or a solid phase. The term “solid phase” describes a non-aqueous matrix to which a protein of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149. The term also encompasses beads or chips that may be suspended in solution.

Proteins herein also encompass antibodies.

Exemplary Antibodies

Antibodies are typically directed against an “antigen” of interest. An antibody that is “directed against” or “specifically binds to” or is “specific for” a given antigen is one that binds to that particular antigen without substantially binding to any other polypeptide or polypeptide epitope. An antibody that is “directed against” or “specifically binds to” or is “specific for” a particular polypeptide or an epitope on a particular polypeptide antigen is one that binds to that particular polypeptide or epitope on a particular polypeptide antigen without substantially binding to any other polypeptide or polypeptide epitope.

Preferably, the antigen is a biologically important molecule and administration of the antibody to a mammal suffering from a disease or disorder can result in a therapeutic benefit in that mammal. Antibodies directed against both protein antigens and non-protein antigens (such as tumor-associated glycolipid antigens; see U.S. Pat. No. 5,091,178) are contemplated. Where the antigen is a protein, it may be a transmembrane molecule (e.g., receptor) or ligand such as a growth factor. Exemplary antigens include those proteins discussed above. Exemplary molecular targets for antibodies encompassed by the present invention include CD polypeptides such as CD3, CD4, CD8, CD19, CD20 and CD34; members of the HER receptor family such as the EGF receptor (RER1), HER2, HER3 or HER4 receptor; cell adhesion molecules such as LFA-1, Mac1, p150,95, VLA-4, ICAM-1, VCAM and av/b3 integrin including either a or b subunits thereof (e.g., anti-CD11a, anti-CD18 or anti-CD11b antibodies); growth factors such as VEGF; IgE; blood group antigens; flk2/flt3 receptor; obesity (OB) receptor; mpl receptor; CTLA-4; polypeptide C etc. Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or may be cells which have been transformed by recombinant techniques to express the transmembrane molecule.

Examples of antibodies to be purified herein include, but are not limited to: HER2 antibodies including trastuzumab (HERCEPTIN®) (Carter et al., Proc. Natl. Acad. Sci. USA, 89:4285-4289 (1992), U.S. Pat. No. 5,725,856) and pertuzumab (OMNITARG™) (WO01/00245); CD20 antibodies (see below); IL-8 antibodies (St John et al., Chest, 103:932 (1993), and International Publication No. WO 95/23865); VEGF or VEGF receptor antibodies including humanized and/or affinity matured VEGF antibodies such as the humanized VEGF antibody huA4.6.1 bevacizumab (AVASTIN®) and ranibizumab (LUCENTIS®) (Kim et al., Growth Factors, 7:53-64 (1992), International Publication No. WO 96/30046, and WO 98/45331, published Oct. 15, 1998); PSCA antibodies (WO01/40309); CD11a antibodies including efalizumab (RAPTIVA®) (U.S. Pat. Nos. 6,037,454, 5,622,700, WO 98/23761, Stoppa et al., Transplant Intl. 4:3-7 (1991), and Hourmant et al., Transplantation 58:377-380 (1994)); antibodies that bind IgE including omalizumab (XOLAIR®) (Presta et al., J. Immunol. 151:2623-2632 (1993), and International Publication No. WO 95/19181; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); CD18 antibodies (U.S. Pat. No. 5,622,700, issued Apr. 22, 1997, or as in WO 97/26912, published Jul. 31, 1997); Apo-2 receptor antibody antibodies (WO 98/51793 published Nov. 19, 1998); Tissue Factor (TF) antibodies (European Patent No. 0 420 937 B1 granted Nov. 9, 1994); α₄-α₇ integrin antibodies (WO 98/06248 published Feb. 19, 1998); EGFR antibodies (e.g., chimeric or humanized 225 antibody, cetuximab, ERBUTIX® as in WO 96/40210 published Dec. 19, 1996); CD3 antibodies such as OKT3 (U.S. Pat. No. 4,515,893 issued May 7, 1985); CD25 or Tac antibodies such as CHI-621 (SIMULECT®) and ZENAPAX® (See U.S. Pat. No. 5,693,762 issued Dec. 2, 1997); CD4 antibodies such as the cM-7412 antibody (Choy et al., Arthritis Rheum 39(1):52-56 (1996)); CD52 antibodies such as CAMPATH-1H (ILEX/Berlex) (Riechmann et al., Nature 332:323-337 (1988)); Fc receptor antibodies such as the M22 antibody directed against FcγRI as in Graziano et al., J. Immunol. 155(10):4996-5002 (1995)); carcinoembryonic antigen (CEA) antibodies such as hMN-14 (Sharkey et al., Cancer Res. 55(23Suppl): 5935s-5945s (1995)); antibodies directed against breast epithelial cells including huBrE-3, hu-Mc 3 and CHL6 (Ceriani et al., Cancer Res. 55(23): 5852s-5856s (1995); and Richman et al., Cancer Res. 55(23 Supp): 5916s-5920s (1995)); antibodies that bind to colon carcinoma cells such as C242 (Litton et al., Eur J. Immunol. 26(1):1-9 (1996)); CD38 antibodies, e.g., AT 13/5 (Ellis et al., J. Immunol. 155(2):925-937 (1995)); CD33 antibodies such as Hu M195 (Jurcic et al., Cancer Res 55(23 Suppl.):5908s-5910s (1995)) and CMA-676 or CDP771; EpCAM antibodies such as 17-1A (PANOREX®); GpIIb/IIIa antibodies such as abciximab or c7E3 Fab (REOPRO®); RSV antibodies such as MEDI-493 (SYNAGIS®); CMV antibodies such as PROTOVIR®; HIV antibodies such as PRO542; hepatitis antibodies such as the Hep B antibody OSTAVIR®; CA125 antibody including anti-MUC16 (WO2007/001851; Yin, B W T and Lloyd, KO, J. Biol. Chem. 276:27371-27375 (2001)) and OvaRex; idiotypic GD3 epitope antibody BEC2; αvβ3 antibody (e.g., VITAXIN®; Medimmune); human renal cell carcinoma antibody such as ch-G250; ING-1; anti-human 17-1 An antibody (3622W94); anti-human colorectal tumor antibody (A33); anti-human melanoma antibody R24 directed against GD3 ganglioside; anti-human squamous-cell carcinoma (SF-25); human leukocyte antigen (HLA) antibody such as Smart ID10 and the anti-HLA DR antibody Oncolym (Lym-1); CD37 antibody such as TRU 016 (Trubion); IL-21 antibody (Zymogenetics/Novo Nordisk); anti-B cell antibody (Imferon); B cell targeting MAb (Immunogen/Aventis); 1D09C3 (Morphosys/GPC); LymphoRad 131 (HGS); Lym-1 antibody, such as Lym-1Y-90 (USC) or anti-Lym-1 Oncolym (USC/Peregrine); LIF226 (Enhanced Lifesci.); BAFF antibody (e.g., WO 03/33658); BAFF receptor antibody (see e.g., WO 02/24909); BR3 antibody; Blys antibody such as belimumab; LYMPHOSTAT-B™; ISF154 (UCSD/Roche/Tragen); gomilixima (Idec 152; Biogen Idec); IL-6 receptor antibody such as atlizumab (ACTEMRA™; Chugai/Roche); IL-15 antibody such as HuMax-Il-15 (Genmab/Amgen); chemokine receptor antibody, such as a CCR2 antibody (e.g., MLN1202; Millennium); anti-complement antibody, such as C5 antibody (e.g., eculizumab, 5G1.1; Alexion); oral formulation of human immunoglobulin (e.g., IgPO; Protein Therapeutics); IL-12 antibody such as ABT-874 (CAT/Abbott); Teneliximab (BMS-224818; BMS); CD40 antibodies, including S2C6 and humanized variants thereof (WO00/75348) and TNX 100 (Chiron/Tanox); TNF-α antibodies including cA2 or infliximab (REMICADE®), CDP571, MAK-195, adalimumab (HUMIRA™), pegylated TNF-α antibody fragment such as CDP-870 (Celltech), D2E7 (Knoll), anti-TNF-α polyclonal antibody (e.g., PassTNF; Verigen); CD22 antibodies such as LL2 or epratuzumab (LYMPHOCIDE®; Immunomedics), including epratuzumab Y-90 and epratzumab I-131, Abiogen's CD22 antibody (Abiogen, Italy), CMC 544 (Wyeth/Celltech), combotox (UT Southwestern), BL22 (NIH), and LympoScan Tc99 (Immunomedics).

Examples of CD20 antibodies include: “C2B8,” which is now called “rituximab” (“RITUXAN®”) (U.S. Pat. No. 5,736,137); the yttrium-[90]-labelled 2B8 murine antibody designated “Y2B8” or “Ibritumomab Tiuxetan” (ZEVALIN®) commercially available from DEC Pharmaceuticals, Inc. (U.S. Pat. No. 5,736,137; 2B8 deposited with ATCC under accession no. HB11388 on Jun. 22, 1993); murine IgG2a “B1,” also called “Tositumomab,” optionally labelled with ¹³¹I to generate the “131I-B1” or “iodine I131 tositumomab” antibody (BEXXAR™) commercially available from Corixa (see, also, U.S. Pat. No. 5,595,721); murine monoclonal antibody “1F5” (Press et al., Blood 69(2):584-591 (1987)) and variants thereof including “framework patched” or humanized 1F5 (WO 2003/002607, Leung, S.; ATCC deposit HB-96450); murine 2H7 and chimeric 2H7 antibody (U.S. Pat. No. 5,677,180); humanized 2H7 (WO 2004/056312, Lowman et al.,); 2F2 (HuMax-CD20), a fully human, high-affinity antibody targeted at the CD20 molecule in the cell membrane of B-cells (Genmab, Denmark; see, for example, Glennie and van de Winkel, Drug Discovery Today 8: 503-510 (2003) and Cragg et al., Blood 101: 1045-1052 (2003); WO 2004/035607; US2004/0167319); the human monoclonal antibodies set forth in WO 2004/035607 and US2004/0167319 (Teeling et al.,); the antibodies having complex N-glycoside-linked sugar chains bound to the Fc region described in US 2004/0093621 (Shitara et al.,); monoclonal antibodies and antigen-binding fragments binding to CD20 (WO 2005/000901, Tedder et al.,) such as HB20-3, HB20-4, HB20-25, and MB20-11; CD20 binding molecules such as the AME series of antibodies, e.g., AME 33 antibodies as set forth in WO 2004/103404 and US2005/0025764 (Watkins et al., Eli Lilly/Applied Molecular Evolution, AME); CD20 binding molecules such as those described in US 2005/0025764 (Watkins et al.,); A20 antibody or variants thereof such as chimeric or humanized A20 antibody (cA20, hA20, respectively) or IMMU-106 (US 2003/0219433, Immunomedics); CD20-binding antibodies, including epitope-depleted Leu-16, 1H4, or 2B8, optionally conjugated with IL-2, as in US 2005/0069545A1 and WO 2005/16969 (Carr et al.,); bispecific antibody that binds CD22 and CD20, for example, hLL2xhA20 (WO2005/14618, Chang et al.,); monoclonal antibodies L27, G28-2, 93-1B3, B-C1 or NU-B2 available from the International Leukocyte Typing Workshop (Valentine et al., In: Leukocyte Typing III (McMichael, Ed., p. 440, Oxford University Press (1987)); 1H4 (Haisma et al., Blood 92:184 (1998)); anti-CD20 auristatin E conjugate (Seattle Genetics); anti-CD20-IL2 (EMD/Biovation/City of Hope); anti-CD20 MAb therapy (EpiCyte); anti-CD20 antibody TRU 015 (Trubion).

Exemplary Antibody Structures

A basic 4-chain antibody unit is a heterotetrameric glycoprotein composed of two identical light (L) chains and two identical heavy (H) chains. An IgM antibody consists of 5 of the basic heterotetramer units along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 Daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, δ, ε, γ. and μ, respectively. The γ and α classes are further divided into subclasses on the basis of relatively minor differences in the CH sequence and function, e.g., humans express the following subclasses: IgG1, IgG2, IgG3, IgG4, IgA1 and IgA2.

The term “variable region” or “variable domain” or “V domain” or “V region” refers to the fact that certain segments of the heavy and light chains differ extensively in sequence among antibodies. The V domain mediates antigen binding and defines the specificity of a particular antibody for its particular antigen. However, the variability is not evenly distributed across the entire span of the variable domains. Instead, the V regions consist of relatively invariant stretches called framework regions (FRs) of about 15-30 amino acid residues separated by shorter regions of extreme variability called “hypervariable regions” (HVRs) or sometimes “complementarity determining regions” (CDRs) that are each approximately 9-12 amino acid residues in length. The variable domains of native heavy and light chains each comprise four FRs, largely adopting a (3-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen binding site of antibodies (see Kabat et al., Sequences of Proteins of Immunological Interest, 5th Ed. Public Health Service, National Institutes of Health, Bethesda, Md. (1991). The constant domains are not involved directly in binding an antibody to an antigen, but exhibit various effector functions, such as participation of the antibody dependent cellular cytotoxicity (ADCC).

The term “hypervariable region” (also known as “complementarity determining regions” or CDRs) when used herein refers to the amino acid residues of an antibody which are (usually three or four short regions of extreme sequence variability) within the V-region domain of an immunoglobulin which form the antigen-binding site and are the main determinants of antigen specificity. There are at least two methods for identifying the CDR residues: (1) An approach based on cross-species sequence variability (i.e., Kabat et al., Sequences of Proteins of Immunological Interest (National Institute of Health, Bethesda, M S 1991); and (2) An approach based on crystallographic studies of antigen-antibody complexes (Chothia, C. et al., J. Mol. Biol. 196: 901-917 (1987)). However, to the extent that two residue identification techniques define regions of overlapping, but not identical regions, they can be combined to define a hybrid CDR.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations, deamidation) that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256: 495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is (are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)). Chimeric antibodies of interest herein include “primitized” antibodies comprising variable domain antigen-binding sequences derived from a non-human primate (e.g., Old World Monkey, Ape etc.) and human content region sequences.

An “intact” or “full length” antibody is one which comprises an antigen-binding site as well as a CL and at least the heavy chain domains, C_{H}1, C_{H}2 and C_{H}3. The constant domains may be native sequence constant domains (e.g., human native sequence constant domains) or amino acid sequence variants thereof. Preferably, the intact antibody has one or more effector functions.

The term “antibody” includes “antibody fragments” and “antigen binding fragments.” An “antibody fragment” or “antigen binding fragment” comprises a portion of an intact antibody that includes the antigen binding portion and/or the variable region of the intact antibody, and that binds specifically to the antigen. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment or “Fc” comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

“Humanized” forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) of mostly human sequences, which contain minimal sequence derived from non-human immunoglobulin. For the most part, humanized antibodies are human immunoglobulins (recipient antibody) in which residues from a hypervariable region (also CDR) of the recipient are replaced by residues from a hypervariable region of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). “Human” or “fully human” antibodies include those that contain framework and constant domain sequences found in human antibodies.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM. The Ig fusions preferably include the substitution of a domain of a polypeptide or antibody described herein in the place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

Protein Formulations and Additional Excipients

The disclosure herein relates to particular protein formulations, for example for therapeutic use. Formulations herein comprise at least one protein species and at least one cholate surfactant, but may also comprise other excipients or ingredients, as described below. For example, formulations herein may comprise one or more of a pharmaceutically acceptable acid or base, buffers, salts, lyoprotectant (if the formulation is to be lyophilized), sugar, sugar alcohol, amino acid, an additional protein species, diluents, preservatives, polyvalent metal salts, and, in some cases another surfactant.

For example, in some embodiments, formulations may comprise a protein, cholate surfactant, and at least one buffer or salt. In some embodiments, formulations may further comprise one or more stabilizers, such as a sugar, sugar alcohol, amino acid, or polyvalent metal salt, depending on the needs of the protein to be formulated. In some embodiments, formulations may comprise a further surfactant such as a polysorbate, poloxamer, pluronic, Brij, or alkylglycoside surfactant.

A “stabilizer” herein means any added excipient that is added to a formulation to help maintain it in a stable or unchanging state. In some cases, a stabilizer may be added to help prevent aggregation, oxidation, color changes, or the like.

A “pharmaceutically acceptable acid” includes inorganic and organic acids which are non-toxic at the concentration and manner in which they are formulated. For example, suitable inorganic acids include hydrochloric, perchloric, hydrobromic, hydroiodic, nitric, sulfuric, sulfonic, sulfinic, sulfanilic, phosphoric, carbonic, etc. Suitable organic acids include straight and branched-chain alkyl, aromatic, cyclic, cycloaliphatic, arylaliphatic, heterocyclic, saturated, unsaturated, mono, di- and tri-carboxylic, including for example, formic, acetic, 2-hydroxyacetic, trifluoroacetic, phenylacetic, trimethylacetic, t-butyl acetic, anthranilic, propanoic, 2-hydroxypropanoic, 2-oxopropanoic, propandioic, cyclopentanepropionic, cyclopentane propionic, 3-phenylpropionic, butanoic, butandioic, benzoic, 3-(4-hydroxybenzoyl)benzoic, 2-acetoxy-benzoic, ascorbic, cinnamic, lauryl sulfuric, stearic, muconic, mandelic, succinic, embonic, fumaric, malic, maleic, hydroxymaleic, malonic, lactic, citric, tartaric, glycolic, glyconic, gluconic, pyruvic, glyoxalic, oxalic, mesylic, succinic, salicylic, phthalic, palmoic, palmeic, thiocyanic, methanesulphonic, ethanesulphonic, 1,2-ethanedisulfonic, 2-hydroxyethanesulfonic, benzenesulphonic, 4-chorobenzenesulfonic, napthalene-2-sulphonic, p-toluenesulphonic, camphorsulphonic, 4-methylbicyclo[2.2.2]-oct-2-ene-1-carboxylic, glucoheptonic, 4,4′-methylenebis-3-(hydroxy-2-ene-1-carboxylic acid), hydroxynapthoic.

“Pharmaceutically-acceptable bases” include inorganic and organic bases which are non-toxic at the concentration and manner in which they are formulated. For example, suitable bases include those formed from inorganic base forming metals such as lithium, sodium, potassium, magnesium, calcium, ammonium, iron, zinc, copper, manganese, aluminum, N-methylglucamine, morpholine, piperidine and organic non-toxic bases including, primary, secondary and tertiary amine, substituted amines, cyclic amines and basic ion exchange resins, [e.g., N(R′)₄+ (where R′ is independently H or C₁₋₄ alkyl, e.g., ammonium, Tris)], for example, isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic non-toxic bases are isopropylamine, diethylamine, ethanolamine, trimethamine, dicyclohexylamine, choline, and caffeine.

Additional pharmaceutically acceptable acids and bases useable with the present invention include those which are derived from the amino acids, for example, histidine, glycine, phenylalanine, aspartic acid, glutamic acid, lysine and asparagine.

Formulations herein may also include one or more buffers or salts. Buffers and salts include those derived from both acid and base addition salts of the above indicated acids and bases. Specific buffers and/or salts include arginine, histidine, succinate and acetate.

If a formulation is to be lyophilized, a lyoprotectant may be added. A “lyoprotectant” is a molecule which, when combined with a protein of interest, significantly prevents or reduces physicochemical instability of the protein upon lyophilization and subsequent storage. Exemplary lyoprotectants include sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred lyoprotectant are the non-reducing sugars trehalose or sucrose.

The lyoprotectant is added to the pre-lyophilized formulation in a “lyoprotecting amount” which means that, following lyophilization of the protein in the presence of the lyoprotecting amount of the lyoprotectant, the protein essentially retains its physicochemical stability upon lyophilization and storage.

A “pharmaceutically acceptable sugar” is a molecule which, when combined with a protein of interest, significantly prevents or reduces physicochemical instability of the protein upon storage. When the formulation is intended to be lyophilized and then reconstituted, “pharmaceutically acceptable sugars” may also be known as a “lyoprotectant”. Exemplary sugars and their corresponding sugar alcohols includes: an amino acid such as monosodium glutamate or histidine; a methylamine such as betaine; a lyotropic salt such as magnesium sulfate; a polyol such as trihydric or higher molecular weight sugar alcohols, e.g., glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred pharmaceutically-acceptable sugars are the non-reducing sugars trehalose or sucrose.

Pharmaceutically acceptable sugars are added to the formulation in a “protecting amount” (e.g., pre-lyophilization) which means that the protein essentially retains its physicochemical stability during storage (e.g., after reconstitution and storage).

The “diluent” of interest herein is one which is pharmaceutically acceptable (safe and non-toxic for administration to a human) and is useful for the preparation of a liquid formulation, such as a formulation reconstituted after lyophilization. Exemplary diluents include sterile water, bacteriostatic water for injection (BWFI), a pH buffered solution (e.g., phosphate-buffered saline), sterile saline solution, Ringer's solution or dextrose solution. In an alternative embodiment, diluents can include aqueous solutions of salts and/or buffers.

A “preservative” is a compound which can be added to the formulations herein to reduce bacterial activity. The addition of a preservative may, for example, facilitate the production of a multi-use (multiple-dose) formulation. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The formulations described herein may be prepared as reconstituted lyophilized formulations. The proteins or antibodies described herein are lyophilized and then reconstituted to produce the liquid formulations of the invention. In this particular embodiment, after preparation of the protein of interest as described above, a “pre-lyophilized formulation” is produced. The amount of protein present in the pre-lyophilized formulation is determined taking into account the desired dose volumes, mode(s) of administration etc. For example, the starting concentration of an intact antibody can be from about 2 mg/ml to about 50 mg/ml, preferably from about 5 mg/ml to about 40 mg/ml and most preferably from about 20-30 mg/ml.

The protein to be formulated is generally present in solution. For example, in the liquid formulations of the invention, the protein may be present in a pH-buffered solution at a pH from about 4-8, and preferably from about 5-7. The buffer concentration can be from about 1 mM to about 20 mM, alternatively from about 3 mM to about 15 mM, depending, for example, on the buffer and the desired tonicity of the formulation (e.g., of the reconstituted formulation). Exemplary buffers and/or salts are those which are pharmaceutically acceptable and may be created from suitable acids, bases and salts thereof, such as those which are defined under “pharmaceutically acceptable” acids, bases or buffers.

In some embodiments, a lyoprotectant is added to a pre-lyophilized formulation. The amount of lyoprotectant in the pre-lyophilized formulation is generally such that, upon reconstitution, the resulting formulation will be isotonic. However, hypertonic reconstituted formulations may also be suitable. In addition, the amount of lyoprotectant must not be too low such that an unacceptable amount of degradation/aggregation of the protein occurs upon lyophilization. However, exemplary lyoprotectant concentrations in the pre-lyophilized formulation are from about 10 mM to about 400 mM, alternatively from about 30 mM to about 300 mM, alternatively from about 50 mM to about 100 mM. Exemplary lyoprotectants include sugars and sugar alcohols such as sucrose, mannose, trehalose, glucose, sorbitol, mannitol. However, under particular circumstances, certain lyoprotectants may also contribute to an increase in viscosity of the formulation. As such, care should be taken so as to select particular lyoprotectants which minimize or neutralize this effect. Additional lyoprotectants are described above under the definition of “lyoprotectants”, also referred herein as “pharmaceutically-acceptable sugars”.

The ratio of protein to lyoprotectant can vary for each particular protein or antibody and lyoprotectant combination. In the case of an antibody as the protein of choice and a sugar (e.g., sucrose or trehalose) as the lyoprotectant for generating an isotonic reconstituted formulation with a high protein concentration, the molar ratio of lyoprotectant to antibody may be from about 100 to about 1500 moles lyoprotectant to 1 mole antibody, and preferably from about 200 to about 1000 moles of lyoprotectant to 1 mole antibody, for example from about 200 to about 600 moles of lyoprotectant to 1 mole antibody.

A mixture of the lyoprotectant (such as sucrose or trehalose) and a bulking agent (e.g., mannitol or glycine) may be used in the preparation of the pre-lyophilization formulation. The bulking agent may allow for the production of a uniform lyophilized cake without excessive pockets therein etc. Other pharmaceutically acceptable carriers, excipients or stabilizers such as those described in Remington's Pharmaceutical Sciences 16th edition, Osol, A. Ed. (1980) may be included in the pre-lyophilized formulation (and/or the lyophilized formulation and/or the reconstituted formulation) provided that they do not adversely affect the desired characteristics of the formulation. Acceptable carriers, excipients or stabilizers are nontoxic to recipients at the dosages and concentrations employed and include; additional buffering agents; preservatives; co-solvents; antioxidants including ascorbic acid and methionine; chelating agents such as EDTA; metal complexes (e.g., Zn-protein complexes); biodegradable polymers such as polyesters; and/or salt-forming counterions such as sodium.

In the case of a lyophilized formulation, after the protein, optional lyoprotectant and other optional components are mixed together, the formulation is lyophilized. Many different freeze-dryers are available for this purpose such as Hull50™ (Hull, USA) or GT20™ (Leybold-Heraeus, Germany) freeze-dryers. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g., 40-60 hrs). Optionally, a secondary drying stage may also be performed depending upon the desired residual moisture level in the product. The temperature at which the secondary drying is carried out ranges from about 0-40° C., depending primarily on the type and size of container and the type of protein employed. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g., 10-15 hours). The pressure may be the same as that employed during the primary drying step. Freeze-drying conditions can be varied depending on the formulation and vial size.

Prior to administration to the patient, a lyophilized formulation is typically reconstituted with a pharmaceutically acceptable diluent such that the protein concentration in the reconstituted formulation is at least about 50 mg/ml, for example from about 50 mg/ml to about 400 mg/ml, alternatively from about 80 mg/ml to about 300 mg/ml, alternatively from about 90 mg/ml to about 150 mg/ml. Such high protein concentrations in the reconstituted formulation are considered to be particularly useful where subcutaneous delivery of the reconstituted formulation is intended. However, for other routes of administration, such as intravenous administration, lower concentrations of the protein in the reconstituted formulation may be desired (for example from about 5-50 mg/ml, or from about 10-40 mg/ml protein in the reconstituted formulation). In certain embodiments, the protein concentration in the reconstituted formulation is significantly higher than that in the pre-lyophilized formulation. For example, the protein concentration in the reconstituted formulation may be about 2-40 times, alternatively 3-10 times, alternatively 3-6 times (e.g., at least three fold or at least four fold) that of the pre-lyophilized formulation.

Reconstitution generally takes place at a temperature of about 25° C. to ensure complete hydration, although other temperatures may be employed as desired. The time required for reconstitution will depend, e.g., on the type of diluent, amount of excipient(s) and protein. Exemplary diluents include sterile water, bacteriostatic water for injection (BWF), a pH buffered solution (e.g., phosphate-buffered saline), sterile saline solution, Ringer's solution or dextrose solution. The diluent optionally contains a preservative. Exemplary preservatives have been described above, with aromatic alcohols such as benzyl or phenol alcohol being the preferred preservatives. The amount of preservative employed is determined by assessing different preservative concentrations for compatibility with the protein and preservative efficacy testing. For example, if the preservative is an aromatic alcohol (such as benzyl alcohol), it can be present in an amount from about 0.1-2.0% and preferably from about 0.5-1.5%, but most preferably about 1.0-1.2%.

Preferably, the reconstituted formulation has less than 6000 particles per vial which are ≥10 μm in size.

The formulation herein may also contain more than one protein as necessary for the particular indication being treated, preferably those with complementary activities that do not adversely affect the other protein. For example, it may be desirable to provide two or more antibodies which bind to the desired target (e.g., receptor or antigen) in a single formulation. Such proteins are suitably present in combination in amounts that are effective for the purpose intended.

Additional proteins such as albumin (human serum albumin or bovine serum albumin, for example) or an immunoglobulin (an IgG constant region, for example) may be added to further stabilize the protein of interest.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for protein, at about 120° C. for about 30 minutes, for example.

Therapeutic formulations are prepared for storage by mixing the active ingredient having the desired degree of purity with further optional carriers, excipients or stabilizers (Remington's Pharmaceutical Sciences 18th edition, Mack Publishing Co., Easton, Pa. 18042 [1990]). Acceptable carriers, excipients, or stabilizers are nontoxic to recipients at the dosages and concentrations employed, and include buffers, antioxidants including ascorbic acid, methionine, Vitamin E, sodium metabisulfite, preservatives, isotonicifiers, stabilizers, metal complexes (e.g., Zn-protein complexes), and/or chelating agents such as EDTA.

When the therapeutic agent is an antibody fragment, the smallest fragment which specifically binds to the binding domain of the target protein may be preferred. For example, based upon the variable region sequences of an antibody, antibody fragments or even peptide molecules can be designed which retain the ability to bind the target protein sequence. Such peptides can be synthesized chemically and/or produced by recombinant DNA technology (see, e.g., Marasco et al., Proc. Natl. Acad. Sci. USA 90: 7889-7893 [1993]).

Buffers are used to control the pH in a range which optimizes the therapeutic effectiveness, especially if stability is pH dependent. Buffers are preferably present at concentrations ranging from about 50 mM to about 250 mM. Suitable buffering agents for use with the present invention include both organic and inorganic acids and salts thereof. For example, citrate, phosphate, succinate, tartrate, fumarate, gluconate, oxalate, lactate, acetate. Additionally, buffers may be comprised of histidine and trimethylamine salts such as Tris.

Preservatives may be added to retard microbial growth, and are typically present in a range from 0.2%-1.0% (w/v). Suitable preservatives for use with the present invention include octadecyldimethylbenzyl ammonium chloride; hexamethonium chloride; benzalkonium halides (e.g., chloride, bromide, iodide), benzethonium chloride; thimerosal, phenol, butyl or benzyl alcohol; alkyl parabens such as methyl or propyl paraben; catechol; resorcinol; cyclohexanol, 3-pentanol, and m-cresol.

Tonicity agents may also be included, for example, to adjust or maintain the tonicity of a liquid composition. When used with large, charged biomolecules such as proteins and antibodies, such agents may interact with the charged groups of the amino acid side chains, thereby lessening the potential for inter and intra-molecular interactions. Tonicity agents can be present in any amount between 0.1% to 25% by weight, preferably 1 to 5%, taking into account the relative amounts of the other ingredients. Preferred tonicity agents include polyhydric sugar alcohols, preferably trihydric or higher sugar alcohols, such as glycerin, erythritol, arabitol, xylitol, sorbitol and mannitol.

In some formulations herein, an additional surfactant is included. In other formulations herein, only cholate surfactants are included and no other types of surfactants are included.

Examples of additional surfactants include polysorbates, such as polysorbate 20 (PS20) and polysorbate 80 (PS80). Other additional surfactants may include poloxamers and pluronics, such as poloxamer 188 or pluronic F68, or Brij. Other additional surfactants may include alkylglycosides, such as octyl maltoside, decyl maltoside, dodecyl maltoside, or octyl glucoside. More generally, “alkylglycosides” include any sugar joined by a linkage to any hydrophobic alkyl, as is known in the art. The linkage between the hydrophobic alkyl chain and the hydrophilic saccharide can include, among other possibilities, a glycosidic, ester, thioglycosidic, thioester, ether, amide or ureide bond or linkage. Exemplary alkylglycosides are provided, for example, in WO 2011/163458.

Additional excipients include agents which can serve as one or more of the following: (1) bulking agents, (2) solubility enhancers, (3) stabilizers and (4) and agents preventing denaturation or adherence to the container wall. Such excipients include: polyhydric sugar alcohols (enumerated above); amino acids such as alanine, glycine, glutamine, asparagine, histidine, arginine, lysine, ornithine, leucine, 2-phenylalanine, glutamic acid, threonine, etc.; organic sugars or sugar alcohols such as sucrose, lactose, lactitol, trehalose, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol and sodium thiosulfate; low molecular weight proteins such as human serum albumin, bovine serum albumin, gelatin or other immunoglobulins; hydrophilic polymers such as polyvinylpyrrolidone; monosaccharides (e.g., xylose, mannose, fructose, glucose; disaccharides (e.g., lactose, maltose, sucrose); trisaccharides such as raffinose; and polysaccharides such as dextrin or dextran.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 18th edition, supra.

Sustained-release preparations may be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. Microencapsulation of recombinant proteins for sustained release has been successfully performed with human growth hormone (rhGH), interferon- (rhIFN-), interleukin-2, and MN rpg 120. Johnson et al., Nat. Med. 2: 795-799 (1996); Yasuda et al., Biomed. Ther. 27: 1221-1223 (1993); Hora et al., Bio/Technology 8: 755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds., (Plenum Press: New York, 1995), pp. 439-462; WO 97/03692; WO 96/40072; WO 96/07399; and U.S. Pat. No. 5,654,010.

The sustained-release formulations of these proteins may be developed using poly lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer”, in Biodegradable Polymers as Drug Delivery Systems (Marcel Dekker; New York, 1990), M. Chasin and R. Langer (Eds.) pp. 1-41.

While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Liposomal or proteinoid compositions may also be used to formulate the proteins or antibodies disclosed herein. See U.S. Pat. Nos. 4,925,673 and 5,013,556.

Stability of the proteins and antibodies described herein may be enhanced through the use of non-toxic “water-soluble polyvalent metal salts”. Examples include Ca²⁺, Mg²⁺, Zn²⁺, Fe²⁺, Fe³⁺, Cu²⁺, Sn²⁺, Sn³⁺, Al²⁺ and Al³⁺. Example anions that can form water soluble salts with the above polyvalent metal cations include those formed from inorganic acids and/or organic acids. Such water-soluble salts have a solubility in water (at 20° C.) of at least about 20 mg/ml, alternatively at least about 100 mg/ml, alternative at least about 200 mg/ml.

Suitable inorganic acids that can be used to form the “water soluble polyvalent metal salts” include hydrochloric, acetic, sulfuric, nitric, thiocyanic and phosphoric acid. Suitable organic acids that can be used include aliphatic carboxylic acid and aromatic acids. Aliphatic acids within this definition may be defined as saturated or unsaturated C₂₋₉ carboxylic acids (e.g., aliphatic mono-, di- and tri-carboxylic acids). For example, exemplary monocarboxylic acids within this definition include the saturated C₂₋₉ monocarboxylic acids acetic, propionic, butyric, valeric, caproic, enanthic, caprylic pelargonic and capryonic, and the unsaturated C₂₋₉ monocarboxylic acids acrylic, propriolic methacrylic, crotonic and isocrotonic acids. Exemplary dicarboxylic acids include the saturated C₂₋₉ dicarboxylic acids malonic, succinic, glutaric, adipic and pimelic, while unsaturated C₂₋₉ dicarboxylic acids include maleic, fumaric, citraconic and mesaconic acids. Exemplary tricarboxylic acids include the saturated C₂₋₉ tricarboxylic acids tricarballylic and 1,2,3-butanetricarboxylic acid. Additionally, the carboxylic acids of this definition may also contain one or two hydroxyl groups to form hydroxy carboxylic acids. Exemplary hydroxy carboxylic acids include glycolic, lactic, glyceric, tartronic, malic, tartaric and citric acid. Aromatic acids within this definition include benzoic and salicylic acid.

Commonly employed water soluble polyvalent metal salts which may be used to help stabilize the encapsulated polypeptides of this invention include, for example: (1) the inorganic acid metal salts of halides (e.g., zinc chloride, calcium chloride), sulfates, nitrates, phosphates and thiocyanates; (2) the aliphatic carboxylic acid metal salts (e.g., calcium acetate, zinc acetate, calcium propionate, zinc glycolate, calcium lactate, zinc lactate and zinc tartrate); and (3) the aromatic carboxylic acid metal salts of benzoates (e.g., zinc benzoate) and salicylates.

Properties of Formulations

Certain formulations herein comprising cholate surfactants may show a reduced degree of protein aggregates after storage or after stress such as agitation or high temperature storage, either visible aggregates or presence of high molecular weight species (HMWS), compared to a control solution that has not been stored or subjected to stress.

An “agitation-induced aggregation inhibiting” amount of a cholate may be included in some formulations herein. This is the amount of that cholate that detectably inhibits agitation-induced aggregation of a protein as compared to an identically treated protein in the absence of the cholate under a particular set of conditions such as agitation at 100 rpm for 24 hours at room temperature. For example, aggregation in the formulation may be compared to a non-agitated control solution to examine for either visible aggregates or presence of HMWS.

In some embodiments herein, the formulation has one or more of the following properties following agitation-induced aggregation experiments. Such experiments, as described in the examples that follow, may be performed on a suitable laboratory shaking apparatus at a speed such as 100 rpm. Specifically, the formulation may show no visible aggregates after 24 hours of agitation at 100 rpm at room temperature; it may show no more than 2% high molecular weight protein aggregates after 24 hours of agitation at 100 rpm at room temperature; it may show no more than 1% high molecular weight protein aggregates after 24 hours of agitation at 100 rpm at room temperature; and/or high molecular weight protein aggregates in the formulation may not increase by more than 0.2% after 24 hours of agitation at 100 rpm at room temperature compared to a non-agitated control. For example, a simple visual inspection may be used to check for the presence of visible aggregates, either through cloudiness of the solution or the presence of a precipitate. High molecular weight species may be detected, for example, by size exclusion chromatography (SEC). Other means that can detect high molecular weight species or that can separate species in a formulation according to size, charge, hydrophobicity or mass include gel electrophoresis, isoelectric focusing, capillary electrophoresis, chromatography such as ion-exchange chromatography, reversed-phase high performance liquid chromatography, peptide mapping, oligosaccharide mapping, mass spectrometry, ultraviolet absorbance spectroscopy, fluorescence spectroscopy, circular dichroism spectroscopy, isothermal titration calorimetry, differential scanning calorimetry, analytical ultracentrifugation, dynamic light scattering, proteolysis, and cross-linking, turbidity measurement, filter retardation assays, immunological assays, fluorescent dye binding assays, protein-staining assays, microscopy, and detection of aggregates via ELISA or other binding assays.

In some embodiments, if the formulation comprises polysorbate 20 or polysorbate 80, the polysorbate 20 or polysorbate 80 in the formulation remains intact to a larger degree after 2 weeks of storage at 40° C., or alternatively, following treatment with CALB lipase, than a formulation with the same ingredients and concentrations, but without cholate. For example, in some embodiments, addition of, for example, 0.05% to 0.5% cholate surfactant reduces or eliminates the visible precipitation of polysorbate 20 or polysorbate 80 free fatty acids from the formulation after 2 weeks of storage at 40° C., or after CALB lipase treatment. For example, in some embodiments, addition of 0.05% to 0.5% CHAPS reduces or eliminates the visible precipitation of polysorbate 20 or polysorbate 80 free fatty acids from the formulation after 2 weeks of storage at 40° C., or after CALB lipase treatment.

Therapeutic Treatments Utilizing Formulations of the Disclosure

“Treatment” refers to both therapeutic treatment and prophylactic or preventative measures. Those in need of treatment include those already with the disorder as well as those in which the disorder is to be prevented. Treatment includes any alleviation or improvement of a subject, such as reduction of a symptom of the disorder, improvement in the subject's quality of life, as well as stabilization of the disorder, prevention of a worsening of the disclosure, cure, reduction of the risk of recurrence, and the like.

A “subject” and “patient” are used interchangeably and generally refer to a mammal receiving a treatment. “Mammal” for purposes of treatment refers to any animal classified as a mammal, including humans, domestic and farm animals, and zoo, sports, or pet animals, such as dogs, horses, rabbits, cattle, pigs, hamsters, gerbils, mice, ferrets, rats, cats, etc. In some embodiments, the subject is human.

A “disorder” is any condition that would benefit from treatment with the protein. This includes chronic and acute disorders or diseases including those pathological conditions which predispose the mammal to the disorder in question. Non-limiting—examples of disorders to be treated herein include carcinomas and inflammations.

A “therapeutically effective amount” is at least the minimum concentration required to affect a measurable treatment of a particular disorder. Therapeutically effective amounts of known protein drugs are well known in the art, while the effective amounts of proteins hereinafter discovered may be determined by standard techniques which are well within the skill of a skilled artisan, such as an ordinary physician.

Antibodies and other proteins may be formulated in accordance with the present invention in either liquid or lyophilized form. The route of administration is in accordance with known and accepted methods, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, inhalation or by sustained release or extended-release means.

For treatment of disorder, the appropriate dosage of an active agent will depend on the type of disorder to be treated, as defined above, the severity and course of the disorder, whether the agent is administered for preventive or therapeutic purposes, previous therapy, the patient's clinical history and response to the agent, and the discretion of the attending physician. The agent is suitably administered to the patient at one time or over a series of treatments.

The methods herein can be combined with known methods of treatment for a disorder, either as combined or additional treatments steps or as additional components of a therapeutic formulation. Dosages and desired drug concentration of pharmaceutical compositions herein may vary depending on the particular use envisioned.

The formulations of the present invention, including but not limited to liquid formulations that have not been lyophilized and reconstituted formulations, can be administered to a mammal in need of treatment with the protein, for example a human, in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation routes. In some embodiments, the formulations are administered to the mammal by subcutaneous (i.e., beneath the skin) administration. For such purposes, the formulation may be injected using a syringe. However, other devices for administration of the formulation are available such as injection devices (e.g., the Inject-ease™ and Genject™ devices); injector pens (such as the GenPen™); auto-injector devices, needleless devices (e.g., MediJector™ and BioJector™); and subcutaneous patch delivery systems.

In some specific embodiments, the disclosure relates to containers comprising a formulation of the invention. For example, the formulations may be packaged into single-use or multiple-use vials or into kits for a single dose-administration unit. In another embodiment of the invention, an article of manufacture is provided, which includes a container comprising the formulation and which may also provide instructions for its use. Suitable containers include, for example, bottles, vials (e.g., dual chamber vials), syringes (such as single or dual chamber syringes) and test tubes. The container may be formed from a variety of materials such as glass or plastic. Such containers or kits comprise both single or multi-chambered pre-filled syringes. Exemplary pre-filled syringes are available from Vetter GmbH, Ravensburg, Germany. The label, which is on, or associated with, the container holding the formulation may indicate directions for reconstitution and/or use. The label may further indicate that the formulation is useful or intended for subcutaneous administration. The container holding the formulation may be a multi-use vial, which allows for repeat administrations (e.g., from 2-6 administrations). The article of manufacture may further comprise a second container comprising a suitable diluent (e.g., BWFI), for example, for reconstitution of a lyophilized formulation. The article of manufacture may further include other materials desirable from a commercial and user standpoint, including other buffers, diluents, filters, needles, syringes, and package inserts with instructions for use.

The appropriate dosage (“therapeutically effective amount”) of the protein will depend, for example, on the condition to be treated, the severity and course of the condition, whether the protein is administered for preventive or therapeutic purposes, previous therapy, the patient's clinical history and response to the protein, the type of protein used, and the discretion of the attending physician. The protein is suitably administered to the patient at one time or over a series of treatments and may be administered to the patient at any time from diagnosis onwards. The protein may be administered as the sole treatment or in conjunction with other drugs or therapies useful in treating the condition in question.

### EXAMPLES

The invention will be more fully understood by reference to the following examples. They should not, however, be construed as limiting the scope of the invention.

### Example 1: Investigation of Cholate Surfactants to Prevent Aggregation of Proteins/Antibodies

General Methods

Shaking or Agitation-Induced Aggregation

In this set of studies, a buffered solution (20 mM histidine acetate or 200 mM arginine succinate or 20 mM sodium acetate, pH 5.5-5.8) of monoclonal antibodies were subjected to shaking on an arm shaker at 100 rpm, at room temperature. These studies were carried out using 5 mL antibody solution filled in 15 cc glass vials. Samples were withdrawn at regular time intervals and analyzed for size variant distribution using size-exclusion chromatography (SEC). Various surfactants of the class of cholates were evaluated for their effectiveness to prevent protein aggregation during shaking. The surfactants were used at concentrations below or above their respective critical micelle concentration (CMC).

Experiments and Results

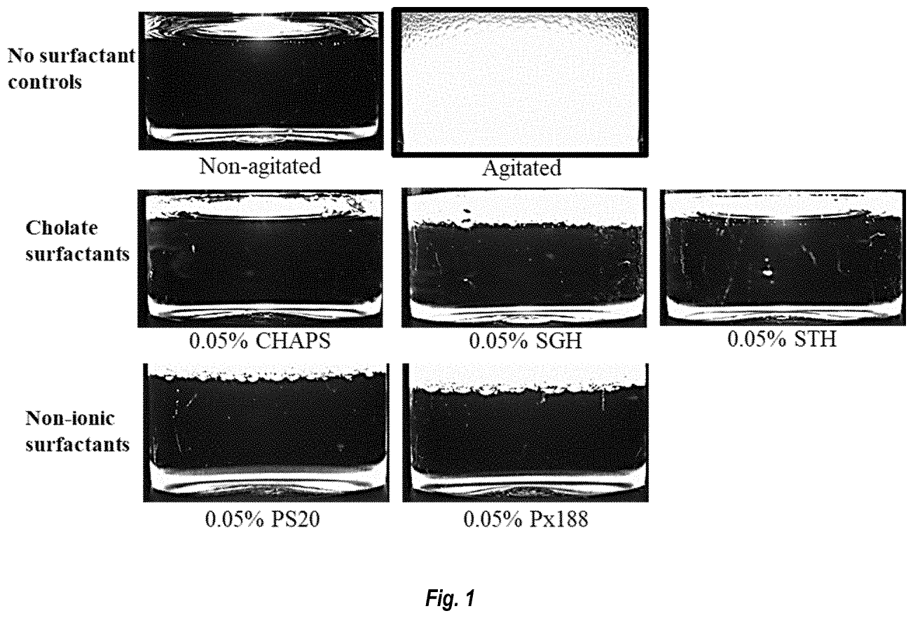

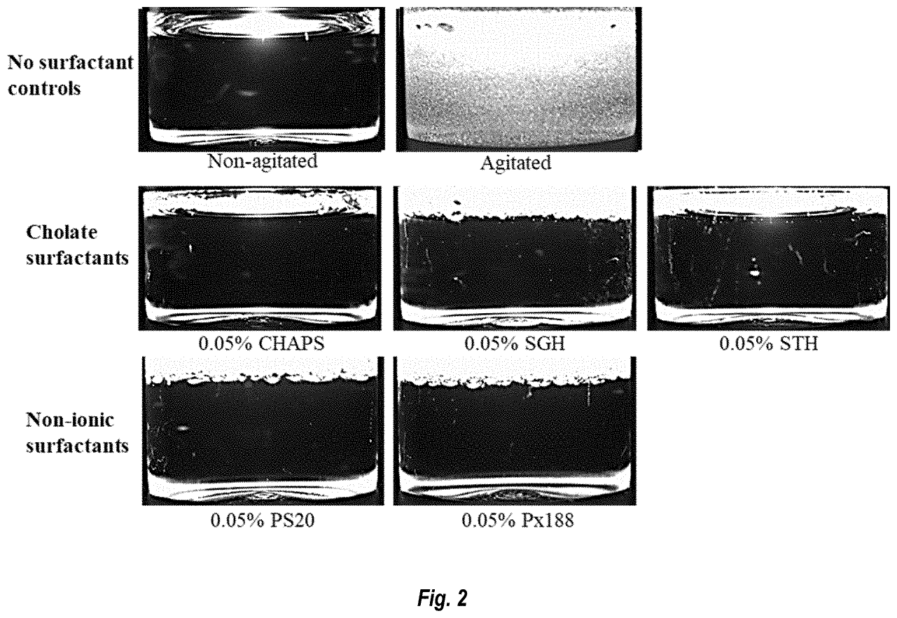

We first investigated whether 0.05% (w/v) of a cholate is sufficient to protect a monoclonal antibody from harsh agitation conditions at low or high ionic strength formulation conditions. We mixed 0.05% (w/v) of a cholate surfactant (CHAPS, SGH, or STH), or control surfactants (PS20 or PX188) with an exemplary monoclonal antibody (anti-PDL1) at 1 mg/mL in a low ionic strength solution of 20 mM histidine acetate and 240 mM sucrose at pH 5.5 or with another exemplary monoclonal antibody (anti-Tryptase) at 1 mg/mL in a high ionic strength solution of 200 mM arginine succinate at pH 5.8. Both solutions were filled in to 15 cc glass vials with a fill volume of 5 mL. Control solutions with the above ingredients but without surfactants were also prepared and filled. The solutions were agitated for 24 hours at ambient temperature in an arm shaker (Glas-Col bench top arm shaker) at 100 rpm. As shown in FIGS. 1 and 2, agitation of no-surfactant control solutions resulted in visibly cloudy solutions, while all other solutions remained visibly clear.

The percentage of HMWS in each solution was also measured by SEC following the 24-hour agitation as a means of determining the extent of protein aggregation. The results are shown in Table 1 and Table 2 below:

TABLE 1
Agitation study - Anti-PDL1 antibody formulated in
low ionic strength buffer - HMWS (%)

| | | Total |
|---|---|---|
| Surfactant Class | Surfactant Type | HMWS % |
| Control | Non-agitated | 1.00 |
| Non-ionic | PS20 | 1.05 |
| | Px188 | 1.03 |
| Zwitterionic | CHAPS | 0.97 |
| Anionic | SGH | 1.87 |
| | STH | 5.93 |
| No surfactant, | agitated, | 23.13 |
| control | no surfactant | |

Table 1 shows the total percentage of HMWS following 24-hour agitation of the 1 mg/mL anti-PDL1 antibody in a low ionic strength solution of 20 mM histidine acetate and 240 mM sucrose at pH 5.5. The anionic SGH and STH surfactants show at least about 2-fold higher total percent HMWS than the control and other surfactant classes. The no surfactant control sample shows significant increase in percent HMWS.

TABLE 2
Agitation study - Anti-Tryptase antibody formulated
in high ionic strength buffer - HMWS (%)

| | | Total |
|---|---|---|
| Surfactant Class | Surfactant Type | HMWS % |
| Control | non agitated | 0.99 |
| Non-ionic | PS20 | 1.03 |
| | PX188 | 1.01 |
| Zwitterionic | CHAPS | 0.96 |
| Anionic | SGH | 0.96 |
| | STH | 0.97 |
| No surfactant, | agitated, | 65.01 |
| control | no surfactant | |

Table 2 shows the total percent HMWS following 24-hour agitation of the 1 mg/mL anti-Tryptase antibody in high ionic strength solution comprising 200 mM arginine succinate at pH 5.8. As shown in Table 2, all surfactants protected low concentration anti-Tryptase antibody from agitation-induced soluble aggregate formation at high ionic strength buffer condition.

Since the zwitterionic CHAPS surfactant performed well in protecting antibody against soluble aggregate formation in both the low ionic strength buffer (Table 1; FIG. 1) and the high ionic strength buffer (Table 2; FIG. 2), it is possible that ionic strength formulation plays a role in making the anionic surfactants (SGH and STH) work effectively. It could be that the presence of high ionic strength in the formulation creates a charge shield so that anionic surfactants such as SGH and STH mainly act as surfactants. To test this hypothesis, we switched the antibodies in the two solutions and repeated the experiments. The results are shown in Table 3 below.

TABLE 3
Agitation study - Anti-Tryptase antibody formulated in high and
low ionic strength buffer (HMWS (%)) (Refer also Table 2)

| | | Total HMWS % | Total HMWS % |
|---|---|---|---|
| Surfactant | Surfactant | (in high ionic | (in low ionic |
| Class | Type | strength) | strength) |
| Control | non agitated | 0.99 | 1.12 |
| Non-ionic | PS20 | 1.03 | 1.10 |
| | Px188 | 1.01 | 1.02 |
| Zwitterionic | CHAPS | 0.96 | 1.05 |
| Anionic | SGH | 0.96 | 8.35 |
| | STH | 0.97 | 28.33 |

Table 3 provides total percent HMWS in each buffer compared to the non-agitated control. The results show that CHAPS, PS20, and PX188 all protect anti-Tryptase from agitation-induced aggregation regardless of ionic strength. The anionic cholate surfactants, SGH and STH, protect anti-Tryptase in high ionic strength formulation from soluble aggregate formation but not at low ionic strength formulation. Similar results are shown for anti-PDL1 in Table 4.

TABLE 4
Agitation study - Anti-PDL1 antibody formulated in high and
low ionic strength buffer - HMWS (%) (Refer also Table 1)

| | | Total HMWS % | Total HMWS % |
|---|---|---|---|
| Surfactant | Surfactant | (in high | (in low |
| Class | Type | ionic strength) | ionic strength) |
| Control | non agitated | 0.88 | 1.00 |
| Non-ionic | PS20 | 1.02 | 1.05 |
| | Px188 | 0.90 | 1.03 |
| Zwitterionic | CHAPS | 1.03 | 0.97 |
| Anionic | SGH | 0.95 | 1.87 |
| | STH | 0.87 | 5.93 |

### Example 2: Test of Impact of Ionic Strength on Aggregation Protection by Cholate Surfactants

Visible particulates following agitation were evaluated in solutions comprising 1 mg/mL of an anti-Tau monoclonal antibody at low ionic strength formulation (20 mM histidine acetate, 240 mM sucrose, pH 5.5) and high ionic strength formulation (20 mM histidine acetate, 272 mM NaCl, pH 5.5) with various cholate surfactants at 0.01%, 0.025%, or 0.05% (w/v) spike-in from a concentrated stock solution of the following surfactants: Sodium Glycocholate Hydrate (SGH), Sodium Taurocholate Hydrate (STH), Sodium Cholate Hydrate (SCH), Sodium Deoxytaurocholate Hydrate (SDTH), Sodium Deoxycholate Hydrate (SDCH), Sodium Chenodeoxycholate Hydrate (SCDCH), CHAPS, and BigCHAP. All formulations were prepared using histidine acetate buffer and the high ionic strength formulation was prepared using sodium chloride in place of arginine succinate. This is to keep the buffer species the same. The purpose is to understand if ionic strength indeed plays a role to preventing HMWS formation but not the buffer species used in the formulation (e.g. arginine).

Whether or not visible particulates were observed under particular conditions is depicted in Tables 5 and 6 below, whilst formation of HMWS compared to the non-agitated controls are shown in Tables 7 and 8 below.

TABLE 5
Agitation study - Anti-Tau formulated in low ionic
strength buffer - Visible particulate analysis

| | | Visible particulate observed? | | |
|---|---|---|---|---|
| Surfactant | Surfactant | (Y/N) | | |
| Class | Type | 0.01% | 0.025% | 0.05% |
| Anionic | SGH | Y | Y | N |
| | STH | Y | Y | N |
| | SCH | Y | N | N |
| | SdTH | Y | Y | N |
| | SdCH | Y | Y | Y |
| | ScdCH | Y | Y | Y |
| Zwitterionic | CHAPS | Y | N | N |
| | BigCHAP | N | N | N |

Y = YES; N = NO

TABLE 6
Agitation study - Anti-Tau formulated in high
ionic strength buffer - Visible particulate analysis

| | | Visible particulate observed? | | |
|---|---|---|---|---|
| Surfactant | Surfactant | (Y/N) | | |
| Class | Type | 0.01% | 0.025% | 0.05% |
| Anionic | SGH | Y | N | N |
| | STH | Y | N | N |
| | SCH | Y | N | N |
| | SdTH | Y | N | N |
| | SdCH | Y | Y | Y |
| | ScdCH | Y | Y | Y |
| Zwitterionic | CHAPS | Y | N | N |
| | BigCHAP | N | N | N |

Y = YES; N = NO

Results in Tables 5 and 6 show that anionic surfactants protect the anti-Tau antibody better from agitation-induced insoluble aggregate formation (visible particle formation) at higher ionic strength formulation when tested at concentrations of 0.025% or 0.05% (w/v). All anionic surfactants and the zwitterionic CHAPS protected antibodies from visible particle formation well at a concentration of at least 0.025% (w/v), with the exception of SdCH and ScdCH, which did not protect anti-Tau from visible particle formation at any of the tested concentrations. BigCHAP protected anti-Tau from agitation induced visible particle formation in all concentrations tested regardless of the ionic strength of the formulation.

TABLE 7
Agitation study - Anti-Tau formulated in low
ionic strength buffer - HMWS (%)

| | | Total HMWS (%) | | | |
|---|---|---|---|---|---|
| | | Control | 0.01% | 0.025% | 0.05% |
| Surfactant | Surfactant | (not | surfac- | surfac- | surfac- |
| Class | Type | agitated) | tant | tant | tant |
| Anionic | SGH | 0.19 | NA | NA | 0.27 |
| | STH | 0.15 | NA | 27.24 | 6.13 |
| | SCH | 0.19 | NA | 0.14 | 0.14 |
| | SdTH | 0.19 | NA | 20.88 | 0.78 |
| | SdCH | NA | NA | NA | NA |
| | ScdCH | NA | NA | NA | NA |
| Zwitterionic | CHAPS | 0.17 | 2.96 | 0.15 | 0.13 |
| | BigCHAP | 0.18 | 2.82 | 0.16 | 0.16 |

NA = protein has completely precipitated out of solution

TABLE 8
Agitation study - Anti-Tau formulated in high
ionic strength buffer - HMWS (%)

| | | Total HMWS (%) | | | |
|---|---|---|---|---|---|
| | | Control | 0.01% | 0.025% | 0.05% |
| Surfactant | Surfactant | (not | surfac- | surfac- | surfac- |
| Class | Type | agitated) | tant | tant | tant |
| Anionic | SGH | 0.73 | 20.41 | 0.74 | 0.74 |
| | STH | 0.72 | NA | 0.93 | 0.73 |
| | SCH | 0.75 | 20.3 | 0.73 | 0.67 |
| | SdTH | 0.84 | NA | 0.83 | 0.82 |
| | SdCH | NA | NA | NA | NA |
| | ScdCH | NA | NA | NA | NA |
| Zwitterionic | CHAPS | 0.83 | 12.65 | 0.85 | 0.85 |
| | BigCHAP | 0.84 | 7.41 | 0.82 | 0.84 |

NA = protein has partially or completely precipitated out of solution

The results in Tables 7 and 8 show that anionic surfactants protect the anti-Tau antibody better from agitation-induced soluble aggregate formation at higher ionic strength formulation when at concentrations of 0.025% or 0.05% (w/v). The zwitterionic CHAPS, BigCHAP and anionic STH protect anti-Tau from soluble aggregate formation in both low and high ionic strength formulation at concentrations of 0.025% (w/v) or 0.05% (w/v). All anionic surfactants protected anti-Tau from soluble aggregate formation well at a concentration of at least 0.025% (w/v), with the exception of SdCH and ScdCH, which did not protect anti-Tau from soluble aggregate formation at any of the tested concentrations.

The results from these studies confirm that it is the ionic strength of the formulation that enabled the anionic surfactants to protect the monoclonal antibodies from agitation induced physical instability but not the type of excipient (sodium chloride arginine) used in the formulation.

### Example 3: Effect of Cholate Surfactants on Protein Charge Heterogeneity (iCIEF)—Agitation Study

Antibody charge variant distribution was evaluated following the agitation experiment described in Example 2 using imaged capillary isoelectric focusing (iCIEF) to determine whether or not cholates have an effect on the relative charge variant distribution of the tested antibodies. The results shown in Tables 9 and 10 below indicate that charge variant distribution was maintained after agitation, and that the cholates, despite them being charged species, did not alter the charge heterogeneity of the antibodies. Anti-PDL1 (1 mg/mL) was incubated with 0.05% surfactant in 20 mM histidine acetate pH 5.5 low ionic strength buffer (Table 9). Anti-Tryptase (1 mg/mL) was incubated with 0.05% surfactant in 200 mM arginine succinate pH 5.8 high ionic strength buffer (Table 10).

TABLE 9
Agitation study - Anti-PDL1 formulated in low ionic
strength buffer - Charge variant assay results (icIEF)

| Surfactant | Surfactant | Acidics | Main Peak | Basics |
|---|---|---|---|---|
| Class | Type | (%) | (%) | (%) |
| Control | Non-agitated | 26.7 | 68.7 | 4.4 |
| Non-ionic | PS20 | 24.8 | 70.2 | 5.0 |
| | Px188 | 26.0 | 69.2 | 4.8 |
| Zwitterionic | CHAPS | 26.1 | 69.0 | 5.0 |
| Anionic | SGH | 26.5 | 68.7 | 4.9 |
| | STH | 27.6 | 67.2 | 5.2 |

TABLE 10
Agitation study - Anti-Tryptase formulated in low ionic
strength buffer - Charge variant assay results (icIEF)

| Surfactant | Surfactant | Acidics | Main Peak | Basics |
|---|---|---|---|---|
| Class | Type | (%) | (%) | (%) |
| Control | Non-agitated | 46.9 | 50.4 | 2.6 |
| Non-ionic | PS20 | 46.5 | 50.4 | 3.1 |
| | Px188 | 47.0 | 50.4 | 2.6 |
| Zwitterionic | CHAPS | 46.8 | 50.6 | 2.6 |
| Anionic | SGH | 47.3 | 50.0 | 2.7 |
| | STH | 45.2 | 46.4 | 8.4* |

*This is unexpected change or value and it could be an assay artifact

Based on the results of Examples 1-3 herein, all zero net charge cholate surfactants prevent soluble aggregate formation at concentrations of 0.05% (w/v) following 24-hour shaking stress. Anionic surfactants (SGH, STH, SCH, and SDTH) appear to prevent soluble aggregate formation better in a high ionic strength buffer such as 200 mM arginine succinate compared to a low ionic strength buffer such as 20 mM histidine acetate (HisOAc). In contrast, the zwitterionic surfactant CHAPS did not show a preference for high or low ionic strength, indicating that ionic strength plays a role in the difference seen with anionic cholate surfactants.

### Example 4: Effect of Cholate Surfactants on Enzymatic Degradation of Polysorbate 20 in Protein Formulations

Upon long-term storage, polysorbate 20 (PS20) can degrade to free fatty acid species (FFA) that can precipitate out of solution, possibly resulting in less protection for proteins in solution as well as the presence of PS20 related particulates forming in a protein formulation or upon reconstitution is not desirable. Such degradation may limit the shelf-life of PS20 containing therapeutic protein formulations. To test whether addition of low concentrations of cholate surfactants impacts polysorbate stability under conditions that mimic PS20 degradation in an accelerated fashion, we spiked in cholates to formulations containing PS20, forced PS20 degradation using lipase, and measured PS20 degradation.

Specifically, solutions containing 5 mg/mL anti-Tryptase antibody formulated in 200 mM arginine succinate, 0.02% (w/v) PS20 at pH 5.8 were mixed with various concentrations of a cholate surfactant, then spiked with 0.04 units/mL CALB and incubated for 12 hours at 5° C. If the presence of cholates in the formulation protects PS20 from degradation to FFAs or solubilizes FFAs that are formed, then visible FFA-related particles should not be observed in the protein solutions. If cholates provide no protection or solubilization, visible FFA-related particles should form to the same degree as protein solutions in which no cholates were added.

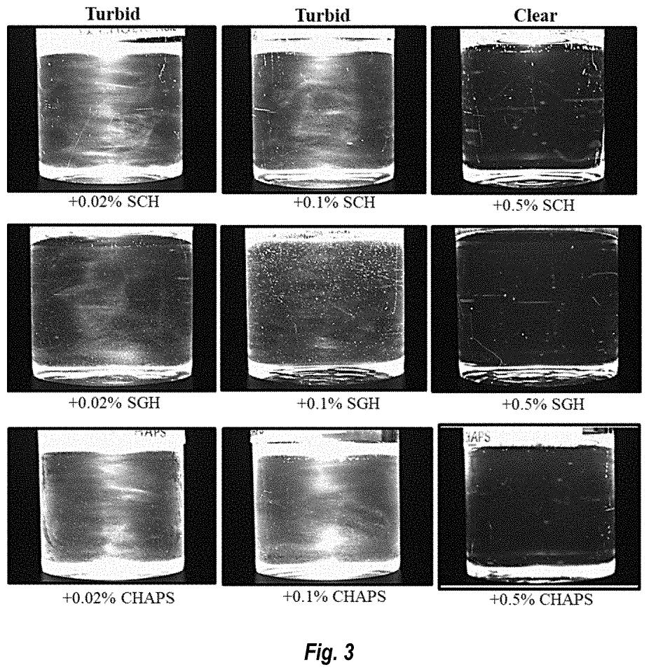

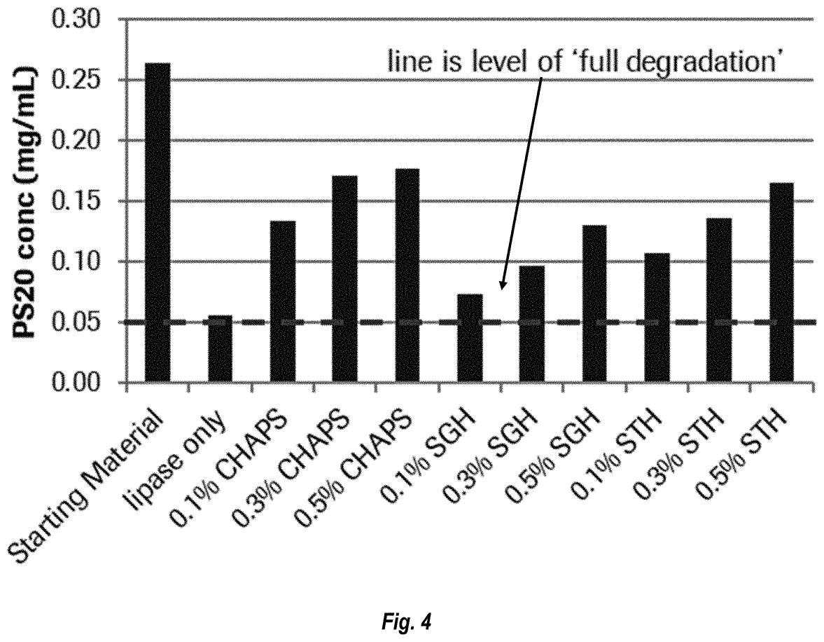

Results from this Experiment are Shown in FIGS. 3 and 4.

The results show that addition of 0.5% SCH, SGH, or CHAPS protects against visible particulate formation in the solutions, while such particulates still form at 0.02% to 0.1% of each added surfactant (FIG. 3).

The amount of PS20 was measured in the starting material to provide a control maximum amount by HPLC-ELSD (Hewitt et al., Journal of Chromatography A. 1215 (2008) 156-160) with standard curve method of 0.25 mg/mL. As shown in FIG. 4, following lipase treatment, the concentration of intact PS20 falls to below 0.1 mg/mL. In contrast, CHAPS, SGH, and STH protected PS20 in the solution from degradation at concentrations of 0.1% to 0.5% (w/v). Specifically, the starting PS20 concentration was measured as just over 0.25 mg/mL, while dropping to 0.05 mg/mL following lipase degradation with no added surfactant. Addition of CHAPS at 0.1% to 0.5% (w/v) allowed the PS20 concentration to remain between about 0.13% and 0.17% (w/v) upon lipase treatment. Addition of SGH at 0.1% to 0.5% (w/v) allowed the PS20 concentration to remain between about 0.06% and 0.13% (w/v) upon lipase treatment. Addition of STH at 0.1% to 0.5% (w/v) allowed the PS20 concentration to remain between just over 0.10% and about 0.16% (w/v) upon lipase treatment.

### Example 5: Effect of Cholate Surfactants on Thermal Degradation of Polysorbate 20 in Protein Formulations

To further test whether cholates can stabilize a PS20-containing protein solution, we added either CHAPS or SGH to a protein solution containing PS20 and subjected the solution to thermal stress for 2 weeks at 40° C. Samples were pulled at Day zero (DO), D7 and D14 and were tested using an intact PS20 HPLC-ELSD quantitation method. The following two monoclonal antibodies were tested with their base formulations: 30 mg/mL anti-PDL1 in 20 mM sodium acetate pH 5.5 and anti-Tryptase in 200 mM arginine succinate pH 5.8. The surfactant spike-in set up and the results for the tests using anti-PDL1 and anti-Tryptase antibodies are provided in Table 11.

TABLE 11
Surfactants co-formulated in to antibody solutions -
Thermal stressed for 2-weeks a 40° C.

| | Change in PS20 Concentration | |
|---|---|---|
| | (mg/mL) | |
| | anti-PDL1 | anti-Tryptase |
| | (30 mg/mL) | (150 mg/ml) |
| | (in low ionic | (in high ionic |
| Sample conditions | strength buffer) | strength buffer) |
| 1:1 (0.05:0.05%) | 0.008 | 0.085 |
| PS20:CHAPS | | |
| 1:2 (0.05:0.1%) | 0.027 | 0.086 |
| PS20:SGH | | |
| PS20 only | 0.028 | 0.10 |

Starting PS20 concentration is 0.05% (w/v)

The data suggest that CHAPS is particularly effective in protecting PS20 against thermal degradation when co-formulated with PS20.

1. A protein formulation comprising a protein and at least one cholate surfactant having a critical micelle concentration (CMC) value of 2.0 mM or greater or of 0.2% (w/v) or greater in water at 25° C.

2. The formulation of claim 1, wherein the protein is an antibody.

3. (canceled)

4. The formulation of claim 1, wherein the cholate surfactant is zwitterionic, nonionic, anionic, or is selected from CHAPS (3-[(3-cholamidopropyl) dimethylammonio]-1-propanesulfonate), SGH (sodium glycocholate hydrate), sodium taurocholate hydrate (STH), sodium cholate hydrate (SCH), SdTH, SdCH, ScdCH, and BigCHAP (N,N′-bis-(3-D-gluconamidopropyl) cholamide).

5. The formulation of claim 4, wherein the formulation comprises CHAPS at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%.

6. (canceled)

7. The formulation of claim 4, wherein the formulation comprises BigCHAP at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%.

8. (canceled)

9. The formulation of claim 4, wherein the formulation comprises SGH, STH, or SCH at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%.

10. (canceled)

11. The formulation of claim 1, wherein the at least one cholate surfactant is present at a concentration that is lower than its CMC value in water at 25° C.

12. The formulation of claim 1, wherein the cholate surfactant is a zwitterionic or nonionic cholate surfactant and wherein the formulation is a low ionic strength formulation.

13. (canceled)

14. The formulation of claim 1, wherein the cholate surfactant is an anionic cholate surfactant, and wherein the formulation is a high ionic strength formulation.

15-16. (canceled)

17. The formulation of claim 1, which has not been subjected to lyophilization.

18. The formulation of claim 17, which is a ready-to-use, liquid formulation.

19. (canceled)

20. The formulation of claim 1, wherein the formulation does not comprise any polysorbate, poloxamer, pluronic, Brij, or alkylglycoside surfactant.

21. (canceled)

22. The formulation of claim 20, wherein the formulation consists essentially of at least one cholate surfactant, at least one protein species, at least one buffer species, and at least one non-surfactant stabilizer.

23. The formulation of claim 1, wherein the formulation further comprises at least one polysorbate or poloxamer.

24. (canceled)

25. The formulation of claim 23, wherein the formulation comprises 0.05% or less of polysorbate 20 or 80.

26. The formulation of claim 25, wherein the formulation does not comprise any surfactant other than the cholate surfactant and the polysorbate 20 or 80.

27. A therapeutic protein formulation, comprising at least (i) one therapeutic protein species, and (ii) a surfactant consisting essentially of
a. CHAPS at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%, wherein the formulation is a low ionic strength formulation;
b. BigCHAP at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%, wherein the formulation is a low ionic strength formulation; or
c. STH, SGH, or SCH at a concentration (w/v) of 0.5% or less, 0.4% or less, 0.3% or less, 0.1% or less, 0.05% or less, 0.04% or less, 0.025% or less, 0.02% or less, 0.01 to 0.5%, 0.01 to 0.1%, 0.01 to 0.05%, or 0.025% to 0.05%, wherein the formulation is a high ionic strength formulation;
and (iii) optionally further comprising one or more of a buffer, a salt, a lyoprotectant, or stabilizer comprising one or more of a sugar, sugar alcohol, amino acid, or other protein species, optionally wherein:
a. The at least one therapeutic protein is an antibody; and/or
b. The formulation is a liquid formulation that is not lyophilized prior to use.

28-32. (canceled)

33. The formulation of claim 1, wherein the formulation has one or more of the following properties:
a. The formulation shows no visible aggregates after 24 hours of agitation at 100 rpm at room temperature;
b. The formulation shows no more than 2% high molecular weight protein aggregates after 24 hours of agitation at 100 rpm at room temperature;
c. The formulation shows no more than 1% high molecular weight protein aggregates after 24 hours of agitation at 100 rpm at room temperature;
d. High molecular weight protein aggregates in the formulation do not increase by more than 0.2% after 24 hours of agitation at 100 rpm at room temperature compared to a non-agitated control;
e. If the formulation comprises polysorbate 20 or polysorbate 80, the polysorbate 20 or polysorbate 80 in the formulation remains intact to a larger degree after 2-weeks storage at 40° C. or after treatment with CALB lipase than a formulation with the same ingredients and concentrations, but without cholate.

34-35. (canceled)

36. A method of making the protein formulation of claim 1, comprising mixing the protein with the at least one cholate surfactant to form a cholate-containing aqueous solution, and optionally further comprising lyophilizing the cholate-containing aqueous solution.

37. A method of inhibiting aggregation of a protein present in an aqueous solution, said method comprising adding to the aqueous solution at least one cholate surfactant having a critical micelle concentration (CMC) value of about 2 mM or greater or 0.2% (w/v) in water at 25° C., at a concentration below its CMC value in water at 25° C., to form a cholate-containing aqueous solution, wherein the protein is optionally an antibody, and wherein the antibody is optionally a monoclonal antibody.

38-41. (canceled)
